SEQUENCE LISTING (22 sequences)

SEQ ID NO: 1 - DNA, 18 bp, Artificial Sequence
  Universal adaptor oligonucleotide
aatacgactcactatagg

SEQ ID NO: 2 - DNA, 19 bp, Artificial Sequence
  universal adaptor oligonucleotide
gctagttattgctcagcgg

SEQ ID NO: 3 - DNA, 18 bp, Artificial Sequence
  universal adaptor oligonucleotide
attaaccctcactaaagg

SEQ ID NO: 4 - DNA, 19 bp, Artificial Sequence
  universal adaptor oligonucleotide
gatttaggtgacactatag

SEQ ID NO: 5 - DNA, 18 bp, Artificial Sequence
  universal adaptor oligonucleotide
tgtaaaacgacggccagt

SEQ ID NO: 6 - DNA, 17 bp, Artificial Sequence
  universal adaptor oligonucleotide
gttttcccagtcacgac

SEQ ID NO: 7 - DNA, 18 bp, Artificial Sequence
  universal adaptor oligonucleotide
caggaaacagctatgacc

SEQ ID NO: 8 - DNA, 21 bp, Artificial Sequence
  universal adaptor oligonucleotide
gactggttccaattgacaagc

SEQ ID NO: 9 - DNA, 21 bp, Artificial Sequence
  universal adaptor oligonucleotide
gcaaatggcattctgacatcc

SEQ ID NO: 10 - DNA, 23 bp, Artificial Sequence
  universal adaptor oligonucleotide
gggctggcaagccacgtttggtg

SEQ ID NO: 11 - DNA, 23 bp, Artificial Sequence
  universal adaptor oligonucleotide
ccgggagctgcatgtgtcagagg

SEQ ID NO: 12 - DNA, 22 bp, Artificial Sequence
  universal adaptor oligonucleotide
aactagaaggcacagtcgaggc

SEQ ID NO: 13 - DNA, 20 bp, Artificial Sequence
  universal adaptor oligonucleotide
cactctcggcatggacgagc

SEQ ID NO: 14 - DNA, 20 bp, Artificial Sequence
  universal adaptor oligonucleotide
tggtgcagatgaacttcagg

SEQ ID NO: 15 - DNA, 20 bp, Artificial Sequence
  universal adaptor oligonucleotide
gttcgaccccgcctcgatcc

SEQ ID NO: 16 - DNA, 21 bp, Artificial Sequence
  universal adaptor oligonucleotide
tgacacacattccacagggtc

SEQ ID NO: 17 - DNA, 19 bp, Artificial Sequence
  universal adaptor oligonucleotide
gcgtgaatgtaagcgtgac

SEQ ID NO: 18 - DNA, 20 bp, Artificial Sequence
  universal adaptor oligonucleotide
ggattattcataccgtccca

SEQ ID NO: 19 - DNA, 20 bp, Artificial Sequence
  universal adaptor oligonucleotide
caaatgtggtatggctgatt

SEQ ID NO: 20 - DNA, 20 bp, Artificial Sequence
  universal adaptor oligonucleotide
atgccatagcatttttatcc

SEQ ID NO: 21 - DNA, 18 bp, Artificial Sequence
  universal adaptor oligonucleotide
gatttaatctgtatcagg

SEQ ID NO: 22 - DNA, 21 bp, Artificial Sequence
  universal adaptor oligonucleotide
cgcaaatgggcggtaggcgtg

(19) United States
(12) Patent Application Publication
(10) Pub. No.: US 2016/0340729 A1
(43) Pub. Date: Nov. 24, 2016

(54) METHODS FOR DEFINING AND PREDICTING IMMUNE RESPONSE TO ALLOGRAFT

(71) Applicants: ADAPTIVE BIOTECHNOLOGIES CORP., Seattle, WA (US); NORTHWESTERN UNIVERSITY, Evanston, IL (US)

(72) Inventors: Ryan O. Emerson, Seattle, WA (US); Ilan Kirsch, Seattle, WA (US); James Mathew, Evanston, IL (US); Joseph Leventhal, Evanston, IL (US)

(21) Appl. No.: 15/110,589

(22) PCT Filed: Jan. 9, 2015
(86) PCT No.: PCT/US15/10904

§ 371 Date: Jul. 8, 2016

Related U.S. Application Data
(60) Provisional application No. 61/925,956, filed on Jan. 10, 2014.
(60) Provisional application No. 62/048,711, filed on Sep. 10, 2014.

(51) Int. Cl. C12Q 1/68

(52) U.S. Cl. CPC C12Q 1/6881; C12Q 2600/118; C12Q 2600/156

(57) ABSTRACT

Methods are provided for predicting and determining a subject's immune response to allograft. Methods include assessing immune response to an allograft by characterizing the diversity and distribution of clones of the adaptive immune repertoire. Methods are also provided for characterizing the adaptive immune response of a subject to an allograft using a mixed lymphocyte reaction culture.

### CROSS REFERENCE TO RELATED APPLICATIONS

This application claims the benefit of U.S. Provisional Application No. 62/048,711, filed on Sep. 10, 2014, and U.S. Provisional Application No. 61/925,956, filed on Jan. 10, 2014, which is each hereby incorporated in its entirety by reference.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ADBS_019_01WO_ST25.txt. The text file is 5 KB, was created on Jan. 9, 2015, and is being submitted electronically via EFS-Web.

### BACKGROUND OF THE INVENTION

Approximately 18,000 kidney transplants are performed in the United States each year. The current prevalence of living kidney transplant recipients is 175,000 (about 400,000 individuals with end stage renal disease (ESRD) are kept alive on dialysis). The 10-year survival of all transplanted kidneys is about 40%. The causes of graft dysfunction resulting in an individual returning to the state of ESRD include hypertension, infection, drug exposure, compromised vascular anatomy, predisposing compromise of the graft pre transplant, age-related renal decline, and, of course, immune rejection. Approximately one third of renal allografts fail for reasons other than rejection. The yearly U.S. health care costs for patients with ESRD are estimated to be over $40 B. Kidney Disease Statistics for the United States. NIH Publication No. 12-3895, June 2012.

When a subject has declining renal function, it is difficult to tell if the decline is due to an allograft rejection or some other factors. Furthermore, when a subject exhibits renal allograft dysfunction, conventional diagnostic analysis requires a direct biopsy of the kidney, a highly invasive procedure that carries the risk of complications and infection Therefore, to achieve a more proactive surveillance monitoring and for the ease of diagnostic sampling, a less invasive approach to diagnosis is needed.

In addition to the difficulties and risk associated with biopsies, biomarkers that could be used to prevent renal compromise in a subject and/or irreversibility of the subject's condition have not yet been identified for transplant-related conditions. Thus, in addition to a less invasive method for diagnosis there is also a need to identify suitable biomarkers to identify whether a subject is at risk for allograft rejection.

The cellular immune response is the most important mediator of transplant rejection and a major barrier to transplant tolerance [1-3]. There is evidence that measuring the immunoglobulin (Ig) and T-cell receptor (TCR) immune response directly at the cellular level and through surrogate biomarkers of immune function can be correlated with allograft rejection. Certain studies have been performed that measure lymphocyte cell count, pattern of infiltration, and CD3 immunohistochemistry as criteria for diagnosing graft rejection in kidney biopsies. Research has focused on identifying markers associated with activated T cell function as indicators of immune mediated rejection. For example, transcript analyses of mRNAs extracted from biopsied allografts have suggested lymphocyte-associated expression changes both in overall profiles (Pavlakis M, Strehlau J, Strom T B: Intragraft T cell receptor transcript expression in human renal allografts. J Am Soc Nephrol 6: 281-285, 1995), as well as in specific genes such as IL-15 (Pavlakis M, Strehlau J, Lipman M, Shapiro M, Malinski W, Strom T B: Intragraft IL-15 transcripts are increased in human renal allograft rejection. Transplantation 62: 543-545, 1996), and CD40 (Zheng X X, Schacter A D, Vasconcellos L, Strehlau J, Tian Y, Shapiro M, Harmon W, Strom T B: Increased CD40 ligand gene expression during human renal and murine islet allograft rejection. Transplantation 65: 1512-1214, 1998).

Cellular immune response is largely mediated by memory T cell populations specific for allo-peptides presented either on allo-MHC (direct antigen presentation) or on self-MHC (indirect antigen presentation) [3-5]. Positive selection in the thymus requiring immature T cells to have some binding affinity for self-HLA means that a significant proportion of mature T cells also have off-target specificity for allo-HLA alleles. Negative selection removes T cells specific for self-peptides presented on self-HLA, but leaves T cells specific for self-peptides presented on allo-HLA [6-12]. The production of the alloreactive T cell repertoire is further complicated by molecular mimicry. In one well-studied example, a public T cell response specific to Epstein Barr Virus (EBV) in the context of HLA-B*08:01 was shown to exhibit cross-reactivity with a self-peptide presented by HLA-B*44:02 [13-16]. These cross-reactive T cells have been observed in HLA-B*08:01/HLA-B*44:02 mismatched lung allografts, suggesting direct clinical relevance for this mode of T cell alloreactivity [17]. Even in individuals with no history of allo-HLA sensitization, viral exposure or vaccine administration can create HLA cross-reactive memory T cells [18-22].

Many studies have identified public and private alloreactive T cell clones that can be primed by a variety of immunogenic events. However, while public T cell clones may play an important role in specific exposures, they represent a very small proportion of the entire T cell repertoire. Investigating private T cell specificities would allow for a much broader view of the alloreactive T cell repertoire, but private T cell responses must be measured anew in each subject.

Accordingly, conventional methods are limited in assessing the potential risk in patients of allograft rejection. Less-invasive approaches are needed to analyze and/or predict the response of transplant patients. There is a need for methods for predicting and assessing the risk of allograft rejection in a subject as well as methods for defining the adaptive immune response of a subject to an allograft.

### SUMMARY OF THE INVENTION

The invention includes methods of defining an alloreactive adaptive immune cell repertoire by obtaining a first sample comprising lymphocytes of a recipient subject at a time point prior to an allograft, and a second sample comprising lymphocytes of a donor subject and obtaining a mixed lymphocyte reaction (MLR) sample comprising a mixture of proliferating lymphocytes from said first and second samples. The method includes generating an adaptive immune profile of adaptive immune cell clones comprising unique rearranged CDR3-encoding region DNA sequences for the first sample and the MLR sample, and identifying one or more alloreactive clones in the adaptive immune profile that are expanded in frequency of occurrence in said MLR sample compared to said first sample.

The method further comprises determining a presence or an absence of the one or more identified alloreactive clones in a post-allograft sample obtained from said recipient subject after the transplant. The method also includes determining a frequency of occurrence of the one or more identified alloreactive clones in a post-allograft sample, wherein the frequency of occurrence of the identified alloreactive clone is predictive of an immune response of the recipient subject to the allograft.

In some embodiments, the method includes determining an adaptive immune profile of adaptive immune cell clones comprises obtaining rearranged DNA templates comprising T cell receptor (TCR) or Immunoglobulin (Ig) CDR3-encoding regions from the lymphocytes in the sample, amplifying the rearranged DNA templates in a single multiplex PCR to produce a plurality of rearranged DNA amplicons, sequencing said plurality of rearranged DNA amplicons to produce a plurality of rearranged DNA sequences, and determining a number of unique rearranged CDR3-encoding DNA sequences in the sample.

The method can comprise determining a frequency of occurrence of each unique rearranged CDR3-encoding DNA sequence in the sample.

In some embodiments, the first sample comprises lymphocytes and the lymphocytes comprise T cells. In one embodiment, the first sample comprises lymphocytes and the lymphocytes comprise B cells.

In other embodiments, the second sample comprises lymphocytes and the lymphocytes comprise T cells. In one embodiment, the second sample comprises lymphocytes and the lymphocytes comprise B cells.

In one aspect, the MLR sample comprises T cells. In another aspect, the MLR sample comprises B cells.

In another embodiment, identifying one or more alloreactive clones comprises identifying a clone that has a frequency of occurrence below a first predetermined threshold in the first sample and has a frequency of occurrence that is greater than a second predetermined threshold in the MLR sample.

In certain aspects, the clone is not observed in the first sample.

In one aspect, the second predetermined threshold is n-fold greater than the first predetermined threshold.

In another embodiment, identifying one or more alloreactive clones comprises identifying a clone that has an n-fold higher frequency of occurrence in the MLR sample than the frequency of occurrence of the clone in the first sample. In one embodiment, n is 2 or greater, or 3 or greater, or 4 or greater, or 5 or greater, or 6 or greater, or 7 or greater or 8 or greater, or 9 or greater, or 10 or greater.

In some embodiments, identifying one or more alloreactive clones comprises identifying a clone that has a statistically significantly higher frequency of occurrence in the MLR sample than in the first sample.

The method also includes characterizing an alloreactive clone as a low-abundance alloreactive clone if the clone has a frequency of occurrence below a predetermined threshold of detection in the sample.

In another embodiment, the method includes characterizing an alloreactive clone as a high-abundance alloreactive clone if the clone has a frequency of occurrence that is greater than a predetermined threshold for a baseline frequency in the sample.

In some embodiments, the method includes characterizing an alloreactive clone as a high-abundance alloreactive clone if the clone has a frequency of occurrence that is statistically significantly greater than a mean frequency of clones in the sample.

In one aspect, the first sample or the second sample comprises a blood sample. In another aspect, the first sample or the second sample comprises a lymphocyte sample. In other aspects, the post-transplant sample comprises a blood sample. In one embodiment, the post-transplant sample comprises a urine sample. In another embodiment, the post-transplant sample comprises a tissue sample.

In certain aspects, the method includes determining that the allograft is rejected based on the frequency of occurrence of at least one identified alloreactive clone in the post-allograft sample.

In other aspects, the method includes determining that the allograft is tolerated based on the frequency of occurrence of at least one identified alloreactive clone in the post-allograft sample.

In another aspect, the method comprises determining a measure of the overlap of alloreactive adaptive immune cell clones between two samples.

In one aspect, the method includes determining a treatment for the recipient subject based on the identified one or more alloreactive clones in the adaptive immune profile.

In one embodiment, the method comprises screening the recipient subject for an allograft based on the identified one or more alloreactive clones in the adaptive immune profile.

In another embodiment, the method includes determining whether an alloreactive adaptive immune cell clone is persistent between two samples.

The method also includes determining whether an alloreactive adaptive immune cell clone is transient between two samples.

Methods of the invention include steps for determining an immune response of a subject undergoing an allograft transplant. In one embodiment determining an immune response is achieved by determining an immune response score. The method can include determining the sequence of a plurality of unique rearranged nucleic acid sequences, each of the plurality of unique rearranged nucleic acid sequences encoding an adaptive immune receptor (AIR) polypeptide, the first sample obtained at a first time point prior to said allograft transplant. The method can include determining a first immune response score for the first sample based on a diversity of the unique rearranged nucleic acid sequences and a distribution of the unique rearranged nucleic acid sequences in the first sample, and determining an immune response of the subject to the allograft transplant based on the first immune response score.

In certain embodiments, the method includes determining the first immune response score comprises quantifying an AIR sequence diversity score for the first sample based on a total number of unique rearranged DNA sequences determined from nucleic acid sequence information from the first sample. In some embodiments, quantifying the AIR sequence diversity score comprises determining a total number of unique clones in the first sample. In another embodiment, determining a first immune response score comprises quantifying an AIR sequence distribution score for the first sample by calculating a frequency of occurrence of each unique rearranged DNA sequence as a percentage of a total number of observed rearranged sequences determined from nucleic acid sequence information from the first sample.

In another embodiment, the method includes determining a first immune response score comprising quantifying an AIR sequence diversity score for the first sample based on a total number of unique rearranged DNA sequences determined from nucleic acid sequence information from the first sample, and quantifying an AIR sequence distribution score for the first sample by calculating a frequency of occurrence of each unique rearranged DNA sequence as a percentage of a total number of observed rearranged sequences determined from nucleic acid sequence information from the first sample.

In certain aspects, the method includes comparing the first immune response score for the first sample to a second immune response score determined for a second sample obtained from said subject at a second time point after the allograft transplant. In some embodiments, the method further includes determining a predicted immune response of the subject to the allograft transplant based on the comparison. In another aspect, the method includes determining that the first immune response score is statistically significantly different from the second immune response score. In yet another aspect, the statistically significant difference is predictive of rejection of the allograft transplant by the subject.

In another embodiment, the method includes determining that the subject has tolerated the allograft transplant based on the comparison of the first immune response score and the second immune response score. In other embodiments, the method comprises determining a frequency of occurrence of one or more clones in said first sample at said first time point and a frequency of occurrence of one or more clones in said second sample at said second time point after said allograft transplant.

In some embodiments, the method includes identifying one or more clones from the second sample that have a frequency of occurrence that is statistically significantly greater than an average frequency of occurrence of the unique rearranged nucleic acid sequences in the second sample. In another embodiment, the method includes identifying one or more clones in the second sample that have a frequency of occurrence that is statistically significantly greater than a top quartile of frequency of occurrence of the unique rearranged nucleic acid sequences in the second sample. In one embodiment, the method includes identifying one or more clones in the second sample that have a frequency of occurrence that is statistically significantly higher than 50% of frequencies of occurrence of the unique rearranged nucleic acid sequences in the second sample. In another embodiment, the method includes determining that the one or more clones is an expanded clone, wherein the expanded clone has increased in frequency of occurrence from a low frequency clone in the first sample to a high frequency clone in the second sample. In one embodiment, the presence of the one or more expanded clones in the second sample is indicative of a rejection of the allograft transplant by the subject.

In another embodiment, the method includes measuring a frequency of occurrence of the one or more expanded clones in subsequent samples obtained from the subject after the allograft transplant. In one embodiment, the first sample and/or the second sample comprise a tissue sample. In another embodiment, the tissue sample comprises a tissue sample from the allograft transplant. In yet another embodiment, the first sample and/or the second sample comprise a circulating blood mononuclear cell fraction. In certain aspects, the first sample and/or the second sample comprise cells collected from urinary sediment.

In another aspect, the nucleic acid sequences comprise genomic DNA sequences. In one aspect, the nucleic acid sequences comprise RNA sequences. In yet another aspect, the nucleic acid sequences comprise complementary DNA (cDNA) sequences.

In some embodiments, the method includes amplifying nucleic acid sequences obtained from a first sample or a second sample comprising lymphoid cells of said subject in a multiplexed polymerase chain reaction (PCR) assay to produce a plurality of amplified nucleic acid sequences using (1) a plurality of AIR V-segment oligonucleotide primers and (2) either a plurality of AIR J-segment oligonucleotide primers or a plurality of AIR C-segment oligonucleotide primers.

In another embodiment, the plurality of AIR V-segment oligonucleotide primers are each independently capable of specifically hybridizing to at least one polynucleotide encoding a mammalian AIR V-region polypeptide, wherein each AIR V-segment oligonucleotide primer comprises a nucleotide sequence of at least 15 contiguous nucleotides that is complementary to at least one functional AIR-encoding gene segment, wherein the plurality of AIR V-segment oligonucleotide primers specifically hybridize to substantially all functional AIR V-encoding gene segments that are present in the first or second samples, wherein the plurality of J-segment oligonucleotide primers are each independently capable of specifically hybridizing to at least one polynucleotide encoding a mammalian AIR J-region polypeptide, wherein each J-segment primer comprises a nucleotide sequence of at least 15 contiguous nucleotides that is complementary to at least one functional AIR J-encoding gene segment, wherein the plurality of J-segment primers specifically hybridize to substantially all functional AIR J-encoding gene segments that are present in the first or second samples; wherein the plurality of C-segment oligonucleotide primers are each independently capable of specifically hybridizing to at least one polynucleotide encoding a mammalian AIR C-region polypeptide, wherein each C-segment primer comprises a nucleotide sequence of at least 15 contiguous nucleotides that is complementary to at least one functional AIR C-encoding gene segment, wherein the plurality of C-segment primers specifically hybridize to substantially all functional AIR C-encoding or gene segments that are present in the first or second samples; and wherein (1) the plurality of AIR V-segment oligonucleotide primers, and (2) either the plurality of AIR J-segment oligonucleotide primers and the plurality of AIR C-segment oligonucleotide primers are capable of promoting amplification in the multiplex PCR of substantially all rearranged AIR CDR3-encoding regions in the first or second samples to produce a plurality of amplified rearranged nucleic acid molecules sufficient to quantify the full diversity of said AIR CDR3-encoding region in the first or second samples.

In another embodiment, each functional AIR V-encoding gene segment comprises a V gene recombination signal sequence (RSS) and each functional AIR J-encoding gene segment comprises a J gene RSS, wherein each amplified rearranged DNA molecule comprises (i) at least 10, 20, 30 or 40 contiguous nucleotides of a sense strand of the AIR V-encoding gene segment, wherein at least 10, 20, 30 or 40 contiguous nucleotides are situated 5′ to the V gene RSS and (ii) at least 10, 20 or 30 contiguous nucleotides of a sense strand of the AIR J-encoding gene segment, wherein at least 10, 20 or 30 contiguous nucleotides are situated 3′ to said J gene RSS.

In yet another embodiment, each amplified rearranged nucleic acid molecule is less than 1500 nucleotides in length. In one aspect, each amplified rearranged nucleic acid molecule is less than 1000 nucleotides in length. In another aspect, each amplified rearranged nucleic acid molecule is less than 600 nucleotides in length. In some aspects, each amplified rearranged nucleic acid molecule is less than 500 nucleotides in length. In other aspects, each amplified rearranged nucleic acid molecule is 400 nucleotides in length. In another aspect, each amplified rearranged nucleic acid molecule is less than 300 nucleotides in length. In one embodiment, each amplified rearranged nucleic acid molecule is less than 200 nucleotides in length. In another embodiment, each amplified rearranged nucleic acid molecule is less than 100 nucleotides in length. In yet another embodiment, each amplified rearranged nucleic acid molecule is between 50-600 nucleotides in length.

In other embodiments, the method includes determining a histocompatibility between a donor subject and a recipient subject using a mixed lymphocyte reaction (MLR). In one embodiment, the method includes identifying clones from a biological sample of the recipient subject using an MLR assay, wherein the clones are predicted to expand in frequency of occurrence after the allograft transplant. In another embodiment, the biological sample comprises a peripheral T-cell population.

In another embodiment, the method includes providing a treatment for the subject based on said determined immune response. In other embodiments, the adaptive immune receptor (AIR) polypeptide is a mammalian AIR polypeptide and is selected from a T cell receptor-gamma (TCRG) polypeptide, a T cell receptor-beta (TCRB) polypeptide, a T cell receptor-alpha (TCRA) polypeptide, a T cell receptor-delta (TCRD) polypeptide, an immunoglobulin heavy-chain (IGH) polypeptide, and an immunoglobulin light-chain (IGL) polypeptide. In another embodiment, the IGH polypeptide is selected from an IgM, an IgA polypeptide, an IgG polypeptide, an IgD polypeptide and an IgE polypeptide. In other embodiments, the IGL polypeptide is selected from an IGL-lambda polypeptide and an IGL-kappa polypeptide. In one embodiment, the mammalian AIR polypeptide is a human AIR polypeptide. In yet another embodiment, the mammalian AIR polypeptide is selected from a non-human primate AIR polypeptide, a rodent AIR polypeptide, a canine AIR polypeptide, a feline AIR polypeptide and an ungulate AIR polypeptide.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG. 1 shows an experimental design for a mixed lymphocyte reaction (MLR) assay followed by high-throughput adaptive immune receptor sequencing. In one example, three pairs of healthy adult subjects were assayed using mixed lymphocyte reaction cultures. For each pair, lymphocytes from a Responder subject (Responder Subject #1) were mixed with inactivated lymphocytes from a Stimulator subject (Stimulator Subject #1) and cultured in duplicate (Cell cultures 1A and 1B). Uncultured freshly isolated PBMC from the Responder as well as proliferating T cell populations from the duplicate cultures were subjected to high-throughput sequencing. Nine samples in total were sequenced across the three pairs of subjects. Three months later, the experiments were repeated to generate nine more samples for high-throughput TCRβ sequencing.

FIG. 2 illustrates an alloreactive cellular subset profile generated in MLR. Bulk MLRs were prepared. The cellular makeup of responder cell populations were delineated at the onset (Day 0) and after 7 days in culture using fluorochrome coupled monoclonal antibodies. The cells were analyzed first by gating on lymphocytes and then after gating either on total CFSE positive responder cells (A: Day 0) or on CFSE diluted proliferating responder cells (B: Day 7).

FIGS. 3A and 3B show T cell clonal frequency among biological replicates of mixed lymphocyte culture. Shown are six scatter plots showing the number of T cells bearing each unique CDR3 sequence in replicate mixed lymphocyte culture experiments performed on three pairs of healthy adult subjects. Each column corresponds to one pair of subjects. FIG. 3A shows plots of T cell clones that were previously observed in a pre-MLR sample of peripheral T cells (high-abundance). FIG. 3B shows plots of T cell clones unobserved in a pre-MLR sample of peripheral T cells (low-abundance). Each point represents a unique T cell clone, and points are plotted at (# of observed T cells+1), so that clones unobserved in one sample are plotted on the axes.

FIGS. 4A-4C show T cell clonal frequency among temporal replicates of mixed lymphocyte culture. FIGS. 4A-4C are three scatter plots of the number of T cells bearing each unique CDR3 sequence in replicate mixed lymphocyte culture experiments performed three months apart on each of three pairs of healthy adult subjects. Considering only T cell clones previously observed in a pre-MLR T cell sample from each time-point and enriched at least ten-fold after mixed lymphocyte culture, each point represents a unique T cell clone and points are plotted at (# of observed T cells+1) so that clones unobserved in one sample are plotted on the axes.

### DETAILED DESCRIPTION OF THE INVENTION

### Overview

The invention comprises methods for prognosis (prediction) of an immune response to an allograft in a subject. The methods also include determining an immune response to an allograft (allograft rejection or toleration) in a subject using high throughput sequencing and calculating an immune response score based on a quantification of diversity and/or clonality of lymphocytes. The invention also includes methods for defining an alloreactive immune cell repertoire for a recipient subject using a mixed lymphocyte reaction.

### Definitions

Terms used in the claims and specification are defined as set forth below unless otherwise specified.

As used herein, adaptive immune receptor (AIR) refers to an immune cell receptor, e.g., a T cell receptor (TCR) or an Immunoglobulin (Ig) receptor found in mammalian cells. In certain embodiments, the adaptive immune receptor is selected from TCRB, TCRG, TCRA, TCRD, IGH, IGK, and IGL.

“Allograft” refers to a graft or a transplant of a tissue or an organ from one individual to another of the same species. The allograft is obtained from a donor subject and given to a recipient subject, e.g., a kidney transplant between two humans.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature.

The term “mammal” as used herein includes both humans and non-humans and include but is not limited to humans, non-human primates, canines, felines, murines, bovines, equines, and porcines.

As used herein a clone is said to be “persistent” when the clone can be identified in two or more samples, or identified at or above a particular threshold between two or more samples. Conversely, as used herein a clone is said to be “transient” when the clone is identified only in one of two or more samples, or is only identified in one of two or more samples at or above a particular threshold.

In some embodiments, as used herein, the term “gene” refers to the segment of DNA involved in producing a polypeptide chain, such as all or a portion of a TCR or Ig polypeptide (e.g., a CDR3-containing polypeptide); it includes regions preceding and following the coding region “leader and trailer” as well as intervening sequences (introns) between individual coding segments (exons), and can also include regulatory elements (e.g., promoters, enhancers, repressor binding sites and the like), and can also include recombination signal sequences (RSSs), as described herein.

The nucleic acids of the present embodiments also referred to herein as polynucleotides, and including oligonucleotides, can be in the form of RNA or in the form of DNA, including cDNA, genomic DNA, and synthetic DNA. The DNA can be double-stranded or single-stranded, and if single stranded can be the coding strand or non-coding (anti-sense) strand. A coding sequence which encodes a TCR or an immunoglobulin or a region thereof (e.g., a V region, a D segment, a J region, a C region, etc.) for use according to the present embodiments can be identical to the coding sequence known in the art for any given TCR or immunoglobulin gene regions or polypeptide domains (e.g., V-region domains, CDR3 domains, etc.), or can be a different coding sequence, which, as a result of the redundancy or degeneracy of the genetic code, encodes the same TCR or immunoglobulin region or polypeptide.

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques can be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to.” By “consisting of” is meant including, and typically limited to, whatever follows the phrase “consisting of” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and can or cannot be present depending upon whether or not they affect the activity or action of the listed elements.

In this specification and the appended claims, the singular forms “a,” “an” and “the” include plural references unless the content clearly dictates otherwise.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

### METHODS OF THE INVENTION

### Cells

A sample containing lymphoid cell DNA (genomic DNA, cDNA or alternatively, messenger RNA) from a subject can be obtained. The subject is a mammalian subject, such as a human.

B cells and T cells can thus be obtained from a biological sample, such as from a variety of tissue and biological fluid samples. These include but are not limited to bone marrow, thymus, lymph glands, lymph nodes, peripheral tissues and blood, or solid tissue samples. Any peripheral tissue can be sampled for the presence of B and T cells and is therefore contemplated for use in the methods described herein. Tissues and biological fluids from which adaptive immune cells, for use in a control adaptive immune cell sample, may be obtained include, but are not limited to skin, epithelial tissues, colon, spleen, a mucosal secretion, oral mucosa, intestinal mucosa, vaginal mucosa or a vaginal secretion, cervical tissue, ganglia, saliva, cerebrospinal fluid (CSF), bone marrow, cord blood, serum, serosal fluid, plasma, lymph, urine, ascites fluid, pleural fluid, pericardial fluid, peritoneal fluid, abdominal fluid, culture medium, conditioned culture medium or lavage fluid. In certain embodiments, adaptive immune cells may be isolated from an apheresis sample. Peripheral blood samples may be obtained by phlebotomy from subjects. Peripheral blood mononuclear cells (PBMC) are isolated by techniques known to those of skill in the art, e.g., by Ficoll-Hypaque® density gradient separation. In certain embodiments, whole PBMCs are used for analysis.

In other embodiments, the sample comprises allograft tissue, a circulating blood mononuclear cell fraction, or cells collected from urinary sediment.

In certain related embodiments, preparations that comprise predominantly lymphocytes (e.g., T and B cells) or that comprise predominantly T cells or predominantly B cells, may be prepared for use as a control adaptive immune cell sample as provided herein, according to established, art-accepted methodologies. In other related embodiments, specific subpopulations of T or B cells may be isolated prior to analysis using the methods described herein. Various methods and commercially available kits for isolating different subpopulations of T and B cells are known in the art and include, but are not limited to, subset selection immunomagnetic bead separation or flow immunocytometric cell sorting using antibodies specific for one or more of any of a variety of known T and B cell surface markers. Illustrative markers include, but are not limited to, one or a combination of CD2, CD3, CD4, CD8, CD14, CD19, CD20, CD25, CD28, CD45RO, CD45RA, CD54, CD62, CD62L, CDw137 (41BB), CD154, GITR, FoxP3, CD54, and CD28.

### Nucleic Acid Extraction

Total genomic DNA can be extracted from cells by methods known to those of skill in the art. Examples include using the QIAamp® DNA blood Mini Kit (QIAGEN®) or a Qiagen DNeasy Blood extraction Kit (Qiagen, Gaithersburg, Md., USA). The approximate mass of a single haploid genome is 3 pg. Preferably, at least 100,000 to 200,000 cells are used for analysis of diversity, i.e., about 0.6 to 1.2 μg DNA from diploid T cells. Using PBMCs as a source, the number of T cells can be estimated to be about 30% of total cells. Alternatively, total nucleic acid can be isolated from cells, including both genomic DNA and mRNA. If diversity is to be measured from mRNA in the nucleic acid extract, the mRNA can be converted to cDNA prior to measurement. This can readily be done by methods of one of ordinary skill.

### Multiplex Quantitative PCR

Multiplex quantitative PCR is described herein and in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, which are incorporated by reference in their entireties. The present methods involve a multiplex PCR method using a set of forward primers that specifically hybridize to V segments and a set of reverse primers that specifically hybridize to the J segments of a TCR or Ig locus, where a multiplex PCR reaction using the primers allows amplification of all the possible VJ (and VDJ) combinations within a given population of T or B cells.

Exemplary V segment and J segment primers are described in U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, which are incorporated by reference in their entireties.

DNA or RNA can be extracted from cells in a sample, such as a sample of blood, lymph, tissue, or other sample from a subject known to contain lymphoid cells, using standard methods or commercially available kits known in the art. In some embodiments, genomic DNA is used. In other embodiments, cDNA is transcribed from mRNA obtained from the cells and then used for multiplex PCR.

A multiplex PCR system can be used to amplify rearranged adaptive immune cell receptor loci from genomic DNA, preferably from a CDR3 region. In certain embodiments, the CDR3 region is amplified from a TCRα, TCRβ, TCRγ or TCRδ CDR3 region or similarly from an IgH or IgL (lambda or kappa) locus. Compositions are provided that comprise a plurality of V-segment and J-segment primers that are capable of promoting amplification in a multiplex polymerase chain reaction (PCR) of substantially all productively rearranged adaptive immune receptor CDR3-encoding regions in the sample for a given class of such receptors (e.g., TCRγ, TCRβ, IgH, etc.) to produce a multiplicity of amplified rearranged DNA molecules from a population of T cells (for TCR) or B cells (for Ig) in the sample. In certain embodiments, primers are designed so that each amplified rearranged DNA molecule in the multiplicity of amplified rearranged DNA molecules is less than 600 nucleotides in length, thereby excluding amplification products from non-rearranged adaptive immune receptor loci.

In the human genome, there are currently believed to be about 70 TCR Vα and about 61 Jα gene segments, about 52 TCR Vβ, about 2 Dβ and about 13 Jβgene segments, about 9 TCR Vγ and about 5 Jγ gene segments, and about 46 immunoglobulin heavy chain (IGH) V_{H}, about 23 D_{H} and about 6 J_{H} gene segments. TCRD has about 8 V gene segments and 4 J segments. TCRA has about 54 V segments and 62 J segments. IgK has about 40 V segments and 5 J segments. IgL has about 35 V segments and 7 J segments. Accordingly, where genomic sequences for these loci are known such that specific molecular probes for each of them can be readily produced, it is believed according to non-limiting theory that the present compositions and methods relate to substantially all (e.g., greater than 90%, 91%, 92%, 93%, 94%, 95%, 96%, 97%, 98% or 99%) of these known and readily detectable adaptive immune receptor V-, D- and J-region encoding gene segments.

The TCR and Ig genes can generate millions of distinct proteins via somatic mutation. Because of this diversity-generating mechanism, the hypervariable complementarity determining regions (CDRs) of these genes can encode sequences that can interact with millions of ligands, and these regions are linked to a constant region that can transmit a signal to the cell indicating binding of the protein's cognate ligand. The adaptive immune system employs several strategies to generate a repertoire of T- and B-cell antigen receptors with sufficient diversity to recognize the universe of potential pathogens. In αβ and γδ T cells, which primarily recognize peptide antigens presented by MHC molecules, most of this receptor diversity is contained within the third complementarity-determining region (CDR3) of the T cell receptor (TCR) α and β chains (or γ and δ chains).

In some embodiments, two pools of primers are used in a single, highly multiplexed PCR reaction. A “forward” pool of primers can include a plurality of V-segment oligonucleotide primers used as “forward” primers and a plurality of J-segment oligonucleotide primers used as “reverse” primers. In certain embodiments, J-segment primers can be used as “forward” primers, and V-segment can be used as “reverse” primers. In some embodiments, there is an oligonucleotide primer that is specific to (e.g., having a nucleotide sequence complementary to a unique sequence region of) each V-region encoding segment (“V segment) in the respective TCR or Ig gene locus. In other embodiments, a primer can hybridize to one or more V segments or J segments, thereby reducing the number of primers required in the multiplex PCR. In certain embodiments, the J-segment primers anneal to a conserved sequence in the joining (“J”) segment.

Each primer can be designed such that a respective amplified DNA segment is obtained that includes a sequence portion of sufficient length to identify each J segment unambiguously based on sequence differences amongst known J-region encoding gene segments in the human genome database, and also to include a sequence portion to which a J-segment-specific primer can anneal for resequencing. This design of V- and J-segment-specific primers enables direct observation of a large fraction of the somatic rearrangements present in the adaptive immune receptor gene repertoire within an individual. This feature in turn enables rapid comparison of the TCR and/or Ig repertoires in individuals pre-transplant and post-transplant, for example.

In one embodiment, the present disclosure provides a plurality of V-segment primers and a plurality of J-segment primers. The plurality of V-segment primers and the plurality of J-segment primers amplify all or substantially all combinations of the V- and J-segments of a rearranged immune receptor locus. In some embodiments, the method provides amplification of substantially all of the rearranged AIR sequences in a lymphoid cell and is capable of quantifying the diversity of the TCR or IG repertoire of at least 10⁶, 10⁵, 10⁴, or 10³ unique rearranged AIR sequences in a sample. “Substantially all combinations” can refer to at least 80%, 85%, 90%, 91%, 92%, 93%, 94%, 95%, 96%, 97%, 98%, 99% or more of all the combinations of the V- and J-segments of a rearranged immune receptor locus. In certain embodiments, the plurality of V-segment primers and the plurality of J-segment primers amplify all of the combinations of the V- and J-segments of a rearranged immune receptor locus.

In general, a multiplex PCR system can use 2, 3, 4, 5, 6, 7, 8, 9, 10, 11, 12, 13, 14, 15, 16, 17, 18, 19, 20, 21, 22, 23, 24, or 25, and in certain embodiments, at least 26, 27, 28, 29, 30, 31, 32, 33, 34, 35, 36, 37, 38, or 39, and in other embodiments 40, 41, 42, 43, 44, 45, 46, 47, 48, 49, 50, 51, 52, 53, 54, 55, 56, 57, 58, 59, 60, 65, 70, 75, 80, 85, or more forward primers, in which each forward primer specifically hybridizes to or is complementary to a sequence corresponding to one or more V region segments. The multiplex PCR system also uses at least 2, 3, 4, 5, 6, or 7, and in certain embodiments, 8, 9, 10, 11, 12 or 13 reverse primers, or 14, 15, 16, 17, 18, 19, 20, 21, 22, 23, 24, or 25 or more primers, in which each reverse primer specifically hybridizes to or is complementary to a sequence corresponding to one or more J region segments. Various combinations of V and J segment primers can be used to amplify the full diversity of TCR and IG sequences in a repertoire. For details on the multiplex PCR system, including primer oligonucleotide sequences for amplifying TCR and IG sequences, see, e.g., Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, which is each incorporated by reference in its entirety.

Oligonucleotides or polynucleotides that are capable of specifically hybridizing or annealing to a target nucleic acid sequence by nucleotide base complementarity can do so under moderate to high stringency conditions. In one embodiment, suitable moderate to high stringency conditions for specific PCR amplification of a target nucleic acid sequence can be between 25 and 80 PCR cycles, with each cycle consisting of a denaturation step (e.g., about 10-30 seconds (s) at greater than about 95° C.), an annealing step (e.g., about 10-30 s at about 60-68° C.), and an extension step (e.g., about 10-60 s at about 60-72° C.), optionally according to certain embodiments with the annealing and extension steps being combined to provide a two-step PCR. As would be recognized by the skilled person, other PCR reagents can be added or changed in the PCR reaction to increase specificity of primer annealing and amplification, such as altering the magnesium concentration, optionally adding DMSO, and/or the use of blocked primers, modified nucleotides, peptide-nucleic acids, and the like.

In certain embodiments, nucleic acid hybridization techniques can be used to assess hybridization specificity of the primers described herein. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

In certain embodiments, the primers are designed not to cross an intron/exon boundary. In some embodiments, the forward primers anneal to the V segments in a region of relatively strong sequence conservation between V segments so as to maximize the conservation of sequence among these primers. Accordingly, this minimizes the potential for differential annealing properties of each primer, and so that the amplified region between V and J primers contains sufficient TCR or Ig V sequence information to identify the specific V gene segment used. In one embodiment, the J segment primers hybridize with a conserved element of the J segment, and have similar annealing strength. In one particular embodiment, the J segment primers anneal to the same conserved framework region motif.

Oligonucleotides (e.g., primers) can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

A primer is preferably a single-stranded DNA. The appropriate length of a primer depends on the intended use of the primer but typically ranges from 6 to 50 nucleotides, or in certain embodiments, from 15-35 nucleotides in length. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

As described herein, primers can incorporate additional features which allow for the detection or immobilization of the primer but do not alter the basic property of the primer, that of acting as a point of initiation of DNA synthesis. For example, primers can contain an additional nucleic acid sequence at the 5′ end, which does not hybridize to the target nucleic acid, but which facilitates cloning, detection, or sequencing of the amplified product. The region of the primer which is sufficiently complementary to the template to hybridize is referred to herein as the hybridizing region.

As used herein, a primer is “specific” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences which contain the target primer binding sites.

In particular embodiments, primers comprise or consist of a nucleic acid of at least about 15 nucleotides long that has the same sequence as, or is substantially complementary to, a contiguous nucleic acid sequence of the target V or J segment. Longer primers, e.g., those of about 16, 17, 18, 19, 20, 21, 22, 23, 24, 25, 26, 27, 28, 29, 30, 31, 32, 33, 34, 35, 36, 37, 38, 39, 40, 45, or 50 nucleotides long that have the same sequence as, or sequence complementary to, a contiguous sequence of the target V or J segment, will also be of use in certain embodiments. Various mismatches (1, 2, 3, or more) to the target sequence can be contemplated in the primers, while preserving complementarity to the target V or J segment. All intermediate lengths of the aforementioned primers are contemplated for use herein. As would be recognized by the skilled person, the primers can have additional sequence added (e.g., nucleotides that cannot be the same as or complementary to the target V or J segment), such as restriction enzyme recognition sites, adaptor sequences for sequencing, bar code sequences, and the like (see e.g., primer sequences provided herein and in the sequence listing). Therefore, the length of the primers can be longer, such as 55, 56, 57, 58, 59, 60, 65, 70, 75, or 80 nucleotides in length or more, depending on the specific use or need. For example, in one embodiment, the forward and reverse primers are both modified at the 5′ end with an adaptor sequence. In some embodiments, the primers comprise a 5′ end sequence that is complimentary to a DNA sequencing oligonucleotide.

Also contemplated are adaptive immune receptor V-segment or J-segment oligonucleotide primer variants that can share a high degree of sequence identity to the oligonucleotide primers. Thus, in these and related embodiments, adaptive immune receptor V-segment or J-segment oligonucleotide primer variants can have substantial identity to the adaptive immune receptor V-segment or J-segment oligonucleotide primer sequences disclosed herein. For example, such oligonucleotide primer variants can comprise at least 70% sequence identity, preferably at least 75%, 80%, 85%, 90%, 91%, 92%, 93%, 94%, 95%, 96%, 97%, 98%, or 99% or higher sequence identity compared to a reference polynucleotide sequence such as the oligonucleotide primer sequences disclosed herein, using the methods described herein (e.g., BLAST analysis using standard parameters). One skilled in this art will recognize that these values can be appropriately adjusted to determine corresponding ability of an oligonucleotide primer variant to anneal to an adaptive immune receptor segment-encoding polynucleotide by taking into account codon degeneracy, reading frame positioning and the like. Typically, oligonucleotide primer variants will contain one or more substitutions, additions, deletions and/or insertions, preferably such that the annealing ability of the variant oligonucleotide is not substantially diminished relative to that of an adaptive immune receptor V-segment or J-segment oligonucleotide primer sequence that is specifically set forth herein. As also noted elsewhere herein, in preferred embodiments adaptive immune receptor V-segment and J-segment oligonucleotide primers are designed to be capable of amplifying a rearranged TCR or IGH sequence that includes the coding region for CDR3.

In some embodiments, the V- and J-segment primers are used to produce a plurality of amplicons from the multiplex PCR reaction. In certain embodiments, the amplicons range in size from 10, 20, 30, 40, 50, 75, 100, 200, 300, 400, 500, 600, 700, 800, 900, 1000, 1100, 1200, 1300, 1400, 1500 to 1600 nucleotides in length. In preferred embodiments, the amplicons have a size between 50-600 nucleotides in length.

According to non-limiting theory, these embodiments exploit current understanding in the art (also described above) that once an adaptive immune cell (e.g., a T or B lymphocyte) has rearranged its adaptive immune receptor-encoding (e.g., TCR or Ig) genes, its progeny cells possess the same adaptive immune receptor-encoding gene rearrangement, thus giving rise to a clonal population that can be uniquely identified by the presence therein of rearranged (e.g., CDR3-encoding) V- and J-gene segments that can be amplified by a specific pairwise combination of V- and J-specific oligonucleotide primers as herein disclosed.

The practice of certain embodiments of the present invention will employ, unless indicated specifically to the contrary, conventional methods in microbiology, molecular biology, biochemistry, molecular genetics, cell biology, virology and immunology techniques that are within the skill of the art, and reference to several of which is made below for the purpose of illustration. Such techniques are explained fully in the literature. See, e.g., Sambrook, et al., Molecular Cloning: A Laboratory Manual (3^{rd} Edition, 2001); Sambrook, et al., Molecular Cloning: A Laboratory Manual (2^{nd} Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, N.Y.); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

In some embodiments, the V segment primers and J segment primers each include a second sequence at the 5′-end of the primer that is not complementary to the target V or J segment. The second sequence can comprise an oligonucleotide having a sequence that is selected from (i) a universal adaptor oligonucleotide sequence, and (ii) a sequencing platform-specific oligonucleotide sequence that is linked to and positioned 5′ to a first universal adaptor oligonucleotide sequence. Examples of universal adaptor oligonucleotide sequences can be pGEX forward and pGEX reverse adaptor sequences, as shown below. Other exemplary universal adaptor sequences are also found in the table below.

TABLE 3
Exemplary Adaptor Sequences

| Adaptor | | SEQ |
|---|---|---|
| (primer) | | ID |
| name | Sequence | NO: |
| T7 Promotor | AATACGACTCACTATAGG | 1 |
| T7 Terminator | GCTAGTTATTGCTCAGCGG | 2 |
| T3 | ATTAACCCTCACTAAAGG | 3 |
| SP6 | GATTTAGGTGACACTATAG | 4 |
| M13F(-21) | TGTAAAACGACGGCCAGT | 5 |
| M13F(-40) | GTTTTCCCAGTCACGAC | 6 |
| M13R Reverse | CAGGAAACAGCTATGACC | 7 |
| AOX1 Forward | GACTGGTTCCAATTGACAAGC | 8 |
| AOX1 Reverse | GCAAATGGCATTCTGACATCC | 9 |
| pGEX Forward | GGGCTGGCAAGCCACGTTTGGTG | 10 |
| (GST 5, pGEX 5′) | | |
| pGEX Reverse | CCGGGAGCTGCATGTGTCAGAGG | 11 |
| (GST 3, pGEX 3′) | | |
| BGH Reverse | AACTAGAAGGCACAGTCGAGGC | 12 |
| GFP (C′ terminal, | CACTCTCGGCATGGACGAGC | 13 |
| CFP, YFP or BFP) | | |
| GFP Reverse | TGGTGCAGATGAACTTCAGG | 14 |
| GAG | GTTCGACCCCGCCTCGATCC | 15 |
| GAG Reverse | TGACACACATTCCACAGGGTC | 16 |
| CYC1 Reverse | GCGTGAATGTAAGCGTGAC | 17 |
| pFastBacF | 5′-d(GGATTATTCATACCGTCCCA)-3′ | 18 |
| pFastBacR | 5′-d(CAAATGTGGTATGGCTGATT)-3′ | 19 |
| pBAD Forward | 5′-d(ATGCCATAGCATTTTTATCC)-3′ | 20 |
| pBAD Reverse | 5′-d(GATTTAATCTGTATCAGG)-3′ | 21 |
| CMV-Forward | 5′-d(CGCAAATGGGCGGTAGGCGTG)-3′ | 22 |

In some embodiments, the resulting amplicons using the V-segment and J-segment primers described above include amplified V and J segments and the universal adaptor oligonucleotide sequences. The universal adaptor sequence can be complementary to an oligonucleotide sequence found in a tailing primer. Tailing primers can be used in a second PCR reaction to generate a second set of amplicons. In some embodiments, tailing primers can have the general formula:

5′-P-S-B-U-3′ (III),

wherein P comprises a sequencing platform-specific oligonucleotide,

wherein S comprises a sequencing platform tag-containing oligonucleotide sequence;

wherein B comprises an oligonucleotide barcode sequence and wherein said oligonucleotide barcode sequence can be used to identify a sample source, and

wherein U comprises a sequence that is complementary to the universal adaptor oligonucleotide sequence or is the same as the universal adaptor oligonucleotide sequence.

Additional description about universal adaptor oligonucleotide sequences, barcodes, and tailing primers are found in PCT/US13/45994, filed on Jun. 14, 2013, which is incorporated by reference in its entirety.

### Amplification Bias Control

Multiplex PCR assays can result in a bias in the total numbers of amplicons produced from a sample, given that certain primer sets are more efficient in amplification than others. To overcome the problem of such biased utilization of subpopulations of amplification primers, methods can be used that provide a template composition for standardizing the amplification efficiencies of the members of an oligonucleotide primer set, where the primer set is capable of amplifying rearranged DNA encoding a plurality of adaptive immune receptors (TCR or Ig) in a biological sample that comprises DNA from lymphoid cells.

In some embodiments, a template composition is used to standardize the various amplification efficiencies of the primer sets. The template composition can comprise a plurality of diverse template oligonucleotides of general formula (I):

5′-U1-B1-V-B2-R-J-B3-U2-3′ (I)

The constituent template oligonucleotides are diverse with respect to the nucleotide sequences of the individual template oligonucleotides. The individual template oligonucleotides can vary in nucleotide sequence considerably from one another as a function of significant sequence variability among the large number of possible TCR or BCR variable (V) and joining (J) region polynucleotides. Sequences of individual template oligonucleotide species can also vary from one another as a function of sequence differences in U1, U2, B (B1, B2 and B3) and R oligonucleotides that are included in a particular template within the diverse plurality of templates.

In certain embodiments, V is a polynucleotide comprising at least 20, 30, 60, 90, 120, 150, 180, or 210, and not more than 1000, 900, 800, 700, 600 or 500 contiguous nucleotides of an adaptive immune receptor variable (V) region encoding gene sequence, or the complement thereof, and in each of the plurality of template oligonucleotide sequences V comprises a unique oligonucleotide sequence.

In some embodiments, J is a polynucleotide comprising at least 15-30, 31-60, 61-90, 91-120, or 120-150, and not more than 600, 500, 400, 300 or 200 contiguous nucleotides of an adaptive immune receptor joining (J) region encoding gene sequence, or the complement thereof, and in each of the plurality of template oligonucleotide sequences J comprises a unique oligonucleotide sequence.

U1 and U2 can be each either nothing or each comprise a universal adaptor oligonucleotide sequence.

B1, B2 and B3 can be each either nothing or each comprise an oligonucleotide B that comprises a first and a second oligonucleotide barcode sequence of 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900 or 1000 contiguous nucleotides (including all integer values therebetween), wherein in each of the plurality of template oligonucleotide sequences B comprises a unique oligonucleotide sequence in which (i) the first barcode sequence uniquely identifies the unique V oligonucleotide sequence of the template oligonucleotide and (ii) the second barcode sequence uniquely identifies the unique J oligonucleotide sequence of the template oligonucleotide.

R can be either nothing or comprises a restriction enzyme recognition site that comprises an oligonucleotide sequence that is absent from V, J, U1, U2, B1, B2 and B3.

Methods are used with the template composition for determining non-uniform nucleic acid amplification potential among members of a set of oligonucleotide amplification primers that are capable of amplifying productively rearranged DNA encoding one or a plurality of adaptive immune receptors in a biological sample that comprises DNA from lymphoid cells of a subject. The method can include the steps of: (a) amplifying DNA of a template composition for standardizing amplification efficiency of an oligonucleotide primer set in a multiplex polymerase chain reaction (PCR) that comprises: (i) the template composition (I) described above, wherein each template oligonucleotide in the plurality of template oligonucleotides is present in a substantially equimolar amount; (ii) an oligonucleotide amplification primer set that is capable of amplifying productively rearranged DNA encoding one or a plurality of adaptive immune receptors in a biological sample that comprises DNA from lymphoid cells of a subject.

In certain embodiments, the primer set can include: (1) in substantially equimolar amounts, a plurality of V-segment oligonucleotide primers that are each independently capable of specifically hybridizing to at least one polynucleotide encoding an adaptive immune receptor V-region polypeptide or to the complement thereof, wherein each V-segment primer comprises a nucleotide sequence of at least 15 contiguous nucleotides that is complementary to at least one functional adaptive immune receptor V region-encoding gene segment and wherein the plurality of V-segment primers specifically hybridize to substantially all functional adaptive immune receptor V region-encoding gene segments that are present in the template composition, and (2) in substantially equimolar amounts, a plurality of J-segment oligonucleotide primers that are each independently capable of specifically hybridizing to at least one polynucleotide encoding an adaptive immune receptor J-region polypeptide or to the complement thereof, wherein each J-segment primer comprises a nucleotide sequence of at least 15 contiguous nucleotides that is complementary to at least one functional adaptive immune receptor J region-encoding gene segment and wherein the plurality of J-segment primers specifically hybridize to substantially all functional adaptive immune receptor J region-encoding gene segments that are present in the template composition.

The V-segment and J-segment oligonucleotide primers are capable of promoting amplification in said multiplex polymerase chain reaction (PCR) of substantially all template oligonucleotides in the template composition to produce a multiplicity of amplified template DNA molecules, said multiplicity of amplified template DNA molecules being sufficient to quantify diversity of the template oligonucleotides in the template composition, and wherein each amplified template DNA molecule in the multiplicity of amplified template DNA molecules is less than 1000, 900, 800, 700, 600, 500, 400, 300, 200, 100, 90, 80 or 70 nucleotides in length.

The method also includes steps of: (b) sequencing all or a sufficient portion of each of said multiplicity of amplified template DNA molecules to determine, for each unique template DNA molecule in said multiplicity of amplified template DNA molecules, (i) a template-specific oligonucleotide DNA sequence and (ii) a relative frequency of occurrence of the template oligonucleotide; and (c) comparing the relative frequency of occurrence for each unique template DNA sequence from said template composition, wherein a non-uniform frequency of occurrence for one or more template DNA sequences indicates non-uniform nucleic acid amplification potential among members of the set of oligonucleotide amplification primers.

Further description about bias control methods are provided in U.S. Provisional Application No. 61/726,489, filed Nov. 14, 2012, U.S. Provisional Application No. 61/644,294, filed on May 8, 2012, and PCT/US2013/040221, filed on May 8, 2013, which are incorporated by reference in their entireties.

Sequencing

Sequencing can be performed using any of a variety of available high throughput single molecule sequencing machines and systems. Illustrative sequence systems include sequence-by-synthesis systems, such as the Illumina Genome Analyzer and associated instruments (Illumina, Inc., San Diego, Calif.), Helicos Genetic Analysis System (Helicos BioSciences Corp., Cambridge, Mass.), Pacific Biosciences PacBio RS (Pacific Biosciences, Menlo Park, Calif.), or other systems having similar capabilities. Sequencing is achieved using a set of sequencing platform-specific oligonucleotides that hybridize to a defined region within the amplified DNA molecules. The sequencing platform-specific oligonucleotides are designed to sequence up amplicons, such that the V- and J-encoding gene segments can be uniquely identified by the sequences that are generated. See, e.g., U.S. appliacation Ser. No. 13/217,126; U.S. application Ser. No. 12/794,507; PCT/US2011/026373; or PCT/US2011/049012, which are each incorporated by reference in its entirety.

PCR Template Abundance Estimation

To estimate the average read coverage per input template in our PCR and sequencing approach, a set of synthetic TCR (or BCR) analogs can be used, comprising each combination of Vβ and Jβ gene segments. These synthetic molecules can be those described in general formula (I) above, and in U.S. Provisional Application No. 61/726,489, filed Nov. 14, 2012, U.S. Provisional Application No. 61/644,294, filed on May 8, 2012, and PCT/US2013/040221, filed on May 8, 2013, which are incorporated by reference in their entireties.

These synthetic molecules can be included in each PCR reaction at very low concentration so that only some types of synthetic template are observed. Using the known concentration of the synthetic template pool, the relationship between the number of observed unique synthetic molecules and the total number of synthetic molecules added to reaction can be simulated (this is very nearly one-to-one at the low concentrations that were used). The synthetic molecules allow calculation for each PCR reaction the mean number of sequencing reads obtained per molecule of PCR template, and an estimation of the number of T cells in the input material bearing each unique TCR rearrangement.

Quantification of an Immune Response Score to Diagnose and/or Determine Response to Allograft Rejection

The invention includes methods to determine an immune response score based on quantification of the diversity and distribution of the adaptive immune receptor (AIR) repertoire within each individual subject's adaptive immune system. The methods described herein can also be used to determine whether an allograft transplant patient has tolerated or rejected the transplant.

In some embodiments, determining an immune response score includes quantifying AIR sequence diversity and AIR sequence distribution as measurements of T or B cell clonality. In some embodiments, quantification of AIR sequence diversity can be determined by quantifying the number of different unique AIR encoding sequences, identified by obtaining distinctive nucleotide sequence information for all rearranged DNA encoding a particular AIR polypeptide in a sample. AIR sequence distribution can be determined by quantifying the frequency of occurrence of each unique rearranged AIR encoding DNA sequence.

Where desired, known estimation or extrapolation methods can be used to determine from the sequence information a repertoire diversity in the subject's entire adaptive immune system. To quantify the relative distribution of each unique sequence, quantitative sequencing methodologies described herein and practiced by those of skill in the art also permit determination of the frequency of occurrence of each particular uniquely rearranged DNA sequence amongst the total number of unique sequences. The AIR sequence distribution can represent the degree of T cell or B cell clonality in a sample from a subject (e.g., quantitative degree of representation, or relative abundance).

Any of a number of known computational tools for processing this distribution parameter can be used to generate distribution values (e.g., the frequency of occurrence of each unique sequence) and diversity values (e.g., the total number of different unique sequences).

A. Adaptive Immune Receptor (AIR) Sequence Diversity

Diversity of unique rearranged TCR or IG encoding nucleic acid sequences from lymphoid cells in a sample reflects the number of different T or B cell clones in a sample from a subject.

Sequence diversity can be determined as the number of clones in a sample of a particular size, such as by direct counting or weighted counting in a sample. Alternatively, the number of different clones in a subject can be estimated based on the number of clones in a subsample.

In another embodiment, an arbitrary cutoff value can be assigned to estimate the number of different “effective” clones, such as counting toward diversity only those clones that account for greater than 0.01% of all T or all B cells in the sample.

In other embodiments, models for weighted or extrapolated diversity determinations can be used to calculate sequence diversity. Examples include entropy models, such as the “unseen species model” (see, e.g., Efron et al., 1976 Biometrika 63:435; Fisher et al., 1943 J. Anim. Ecol. 12:42) or other suitable models as will be known to those familiar with the art.

In some embodiments, AIR diversity can be measured by quantitative sequencing of the total AIR observed sequences in a particular sample. Compositions and methods for quantitative sequencing of rearranged adaptive immune receptor gene sequences and for adaptive immune receptor clonotype determination are described, for example, in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, herein incorporated by reference. Therein can also be found details regarding sequences of PCR amplification oligonucleotide primers and sequencing primers, sequencing of PCR amplification products, processing sequencing data, and uses of measurements of adaptive immune receptor diversity, all of which can be employed for use according to the methods described herein.

In some embodiments, a sequencing program such as Raw HiSeg™ can be used to preprocess sequence data to remove errors in the primary sequence of each read, and to compress the sequence data. A nearest neighbor algorithm can be used to collapse the data into unique sequences by merging closely related sequences, to remove both PCR and sequencing errors.

A diversity score can be rated as “low” when there are a few unique rearranged AIR sequences in the repertoire as compared to the total number of observed rearranged AIR sequences in a sample. In some embodiments, the diversity score is rated as “high” when there is a high number of unique rearranged AIR sequences in the repertoire as compared to the total number of observed rearranged AIR sequences in a sample. The determination of a low or high diversity score or rating can be based on pre-determined thresholds, as can be determined by one of skill in the art.

Other methods for calculating AIR sequence diversity can be used, as known to those of skill in the art. For example, the following works, which are incorporated by reference in their entireties, summarize the current theory and practice of estimating diversity indices from species abundance data, while giving detailed examples of several common embodiments of diversity index measurement. See Anne E. Magurran and Brian J. McGill. 2011. Biological Diversity: Frontiers in Measurement and Assessment. New York: Oxford University Press. Other examples of methods for genetic diversity estimation that can be applied to calculate a diversity score rating can be found in James F. Crow and Motoo Kimura. 2009. An Introduction to Population Genetics Theory. Blackburn Press.

B. Adaptive Immune Receptor (AIR) Sequence Distribution

In some embodiments, the AIR sequence distribution can be calculated to determine an immune response score and to determine the subject's response to an allograft transplant. AIR sequence distribution, such as TCR or IG sequence distribution, refers to the variation among the number of different T cell or B cell clones in a sample, e.g., the number of cells that express an identical TCR or IG. For example, AIR sequence distribution can be determined by quantifying the frequency of occurrence of each unique rearranged AIR encoding DNA sequence, as a percentage of the total number of observed rearranged AIR encoding DNA sequences. The quantified distribution of AIR sequences can be used, optionally along with AIR sequence diversity, to calculate the immune response score of a subject and to diagnose allograft rejection.

In some embodiments, an AIR sequence distribution can be determined by, but not limited to, the following methods: (i) identifying and quantifying at least 1-20 of the most abundant unique rearranged (clonal) AIR sequences in a subject over a time interval, or (ii) by identifying and quantifying the number of unique rearranged (clonal) AIR sequences that are needed to account for a given percentage (e.g., up to 10, 20, 30, 40 or 50%) of the total number of observed rearranged sequences in a sample from a subject.

Other calculations can additionally or alternatively be employed to determine AIR sequence distribution of a sample from a subject and to assign a sequence distribution value to a particular sample for purposes of rating the sample in comparison to a control or another sample with a known immunological status. These can include, for example, determining entropy (i.e., Shannon entropy as typically defined in information theory, which can be normalized to the range [0-1] by dividing by the logarithm of the number of elements in the sample set) or using other known methods to determine one or more modes of distribution (e.g., mean, skewness, kurtosis, etc.). The present methods permit determination of sequence distribution and clonality with a degree of precision not previously possible and permit a variety of prognostic, diagnostic, prescriptive and other capabilities.

C. Immune Response Score Calculations

In some embodiments, an immune response score can be determined from a tissue allograft of a subject using the AIR sequence diversity and AIR sequence distribution scores described above. In one embodiment, the AIR sequence diversity score and the AIR sequence distribution scores are used to calculate an immune response score.

In certain embodiments, the immune response score is calculated as a function of the number of immune cells and difference in measurement of clonality relative to either 1) simultaneously assessed repertoire in the peripheral blood from the subject or 2) a measured clonality from a previous biopsy of the same tissue. An increase in the measurement of clonality from the pre-transplant and post-transplant sample or between the tissue sample and peripheral blood sample indicates a response by the subject's immune system to the transplant, and thus a rejection of the transplant by the subject. The increase in clonality can be measured by a statistically significant difference between the AIR diversity and AIR distribution scores of two samples (pre/post transplant tissue or tissue and peripheral blood samples).

In one embodiment, the clonality (diversity) and frequency of individual clones in the sample are used to determine an immune response calculation for a subject. The presence of one or more “expanded” clones in a sample can indicate an immune response by the subject to the transplant (e.g., rejection of the transplant). Using the methods described above, one can determine the number of unique rearranged TCR or IG sequences (e.g., clones) is identified in the sample and the frequency of each clone in the total number of nucleated cells in the sample. In addition, one can determine the frequency of each clone in the total number of lymphocytes in the sample. These calculations can be used to determine the presence of one or more expanded or dominant clones in the sample, indicating an immune response to the allograft transplant.

In one embodiment, a single sample is assessed for an immune response after an allograft transplant. The sample can be a tissue sample or a blood sample. In one example, a tissue sample can have a total of 999,990 cells (nucleated cells), for example kidney cells, and a total of 10 lymphocyte cells. Of these 10 lymphocytes, 8 can be different clones and 2 are the same clone “A.” The frequency of clone A in the total number of lymphocytes is 2 out of 10, which is 20%. However, clone A has a frequency of occurrence in the total number of nucleated cells of 2 in 1,000,000, which is 0.0002%. Even though clone A has a frequency of occurrence of 20% among the total number of unique clones, the low frequency of occurrence of clone A in the total number of nucleated cells in the sample (0.0002%) indicates a lower likelihood that the subject has had an immune response to the allograft transplant.

In another example, a sample has a total of 800,000 cells, for example kidney cells, and 200,000 lymphocyte cells. Here, a particular clone “A” is present in 40,000 of the 200,000 lymphocyte cells. Clone A represents 20% of the total number of lymphocytes in the sample. In addition, clone A has a frequency of occurrence of 40,000 out of 1,000,000 total nucleated cells in the sample (4%). The remainder of the clones can have significantly lower frequencies of occurrence in the sample. This provides a pattern of distribution (e.g., entropy) where one clone (clone A) is a dominant clone. The pattern of distribution and frequency of occurrence calculations for clone A indicate that the subject has likely experienced an immune response to the allograft tissue.

In another embodiment, the method includes comparing immune response scores or calculations from at least two samples. In some embodiments, the samples can be obtained from the same subject (e.g., pre- and post-transplant). In one embodiment, a first sample is a blood sample, and a second sample is a tissue sample, or vice versa. In other embodiments, the samples are both blood samples. In another embodiment, both samples are tissue samples from the subject. Calculations for the diversity (e.g., number of unique clones) and the distribution (e.g., frequency of occurrence) of each clone in a first sample can be compared to the diversity and distribution of clones in a second sample. Statistically significant differences (or differences above a predetermined threshold) among the diversity and distribution scores of the samples can indicate an immune response in the subject.

### Lymphoid-Mediated Allograft Rejection

Lymphoid cells are one of the cell lineages that infiltrate and become integrated within various tissues as a result of normal physiology. Tissue infiltrating lymphocytes are subject to both qualitative and quantitative changes in response to a variety of inflammatory and oncologic disease states. This phenomenon has been most extensively exploited recently in the recognition of the immunogenic nature of certain malignancies and the attempts to maximize lymphocyte-mediated tumoricidal activity into the development of cancer immunotherapy by either brute force ex vivo quantitative expansion of tumor infiltrating lymphocytes or by qualitative alteration of lymphocyte immune enhancing or suppressing activity.

Lymphoid-mediated allograft rejection is an example of tissue specific lymphocyte infiltration and can be subject to the same types of quantitative and qualitative assessments that are currently being evaluated in oncology. A determination of the number and diversity of tissue infiltrating lymphocytes in transplanted organs or allograft tissue can indicate whether a subject has tolerated or responded negatively to a transplant.

The number and diversity of tissue infiltrating lymphocytes in transplanted organs or allograft tissue of a subject has tolerated or responded negatively to the transplant. Immune score profiling involves quantitative immunohistochemistry to delineate the density, location (distribution), and subtype of lymphocytes within a given tissue. Sequencing the immune repertoire within a given tissue section or sample (either in toto or microdissected), as described in methods above, provides complementary and supplementary information, expanding what is currently a two-dimensional analysis to a study of a volume of tissue, and defining the level of diversity and clonality of the lymphocytes that reside within a tissue sample at a point in time (defined by the biopsy or sample collection) and in some cases, how the profile of diversity and clonality is similar to or distinct from the lymphocyte profile in other tissues (including blood).

Determining Alloreactive Clones Using Mixed Lymphocyte Reaction (MLR) Culture

In some embodiments, a subject's immune response to an allograft can be predicted by using a mixed lymphocyte reaction, which identifies alloreactive clones from the recipient subject.

For example, in a “one-way” MLR, donor cells are made replication incompetent, for example, by irradiation or mitomycin C treatment and are placed in culture with recipient lymphocytes. Donor cells are then mixed with a recipient's lymphocytes. The recipient's lymphocytes can be obtained from a peripheral blood sample, for example. In one embodiment, the culture is maintained for 5-7 days and, an agent that can be used to quantitate cell division (e.g., BuDR) is added toward the end of the incubation. Robust BuDR incorporation is consistent with a proliferative response of the recipient cells to “foreign” antigens (e.g. a different HL-A antigen or antigens) on the surface of the donor cells. Unstimulated and non-specific mitogen stimulated cells serve as controls.

The number and frequency of clones can be measured after the mixed lymphocyte reaction culture. Recipient clones that expand in frequency after the MLR are identified as alloreactive clones. The presence of one or more expanded clones can be indicative of a negative response of the donor's cells and predictive of an allograft rejection.

In another embodiment, the lymphocytes are isolated, and the recipient cells and the donor cells are each labeled with different labels, such as with a fluorescent cell staining dye (i.e., CFSE or PKH26). The recipient and donor cells are then cultured in bulk in culture medium, and after a period of time, the cells are harvested, and the proliferating recipient cells are then sorted.

Each population of cells is then subject to amplification and high-throughput sequencing of the CDR3 region of the TCR or IG locus. These data are then used to calculate an immune response score as described above.

Size of the Alloreactive T Cell or B Cell Repertoire

In some embodiments, to determine the number of T cell clonal lineages involved in the alloreactive T cell response, the number of unique CDR3 sequences observed in the proliferated T cell samples is determined in comparison to uncultured bulk T cells from the same subjects. Alloreactive T cell clones are defined as those observed in at least N number of cells (e.g., at least 10 cells) in the proliferated sample and unobserved in the uncultured T cell sample, or T cells whose frequency in the proliferated sample was at least N-fold higher (e.g., ten-fold higher) than in the uncultured T cell sample. For example, two sets of alloreactive T cell clones can be defined: low-abundance alloreactive clones (below the threshold of detection in the subject's baseline T cell repertoire) and high-abundance alloreactive clones (present at measurable frequency in the subject's baseline T cell repertoire). Similar methods can be applied for measuring the size of the B cell repertoire.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992)

### Example 1

### Diagnosis and/or Prediction of Lymphoid-Mediated Allograft Rejection

Lymphoid-mediated allograft rejection is a type of tissue specific lymphocyte infiltration. A determination of the number and diversity of tissue infiltrating lymphocytes in transplanted organs or allograft tissue can indicate whether a subject has tolerated or responded negatively to a transplant.

In some embodiments, the methods for quantifying an immune response score, as described above, are used to assess whether the subject has tolerated or responded negatively to the transplant. The methods can be performed using samples comprising allograft tissue itself, the circulating blood mononuclear cell fraction, or cells collected from urinary sediment.

An immune response score can be calculated by determining the correspondence and correlation of transplant-reactive clones, comparing the diversity and distribution of the clones in each of the tissues or samples of interest, and using the calculated values for prognostic and clinical significance.

In one embodiment, the method can include the following steps for a given sample:

(i) obtaining nucleic acid sequence information generated from one or more samples comprising nucleic acids from lymphoid cells of a subject, wherein said nucleic acid sequence information comprising sequences for a plurality of unique rearranged nucleic acid sequences, each of said plurality of unique rearranged nucleic acid sequences encoding an AIR polypeptide, said one or more samples obtained from the subject at one or more time points (e.g., before and after an allograft);

(ii) determining a total number of observed rearranged sequences in the sample;

(iii) determining a total number of unique rearranged DNA sequences in the sample;

(iv) quantifying an AIR sequence diversity score for the one or more samples based on the total number of unique rearranged DNA sequences;

(v) quantifying an AIR sequence distribution score for said one or more samples by calculating a frequency of occurrence of each unique rearranged DNA sequence as a percentage of said total number of observed rearranged sequences in said one or more samples;

(vi) determining an immune response score for the sample based on a diversity of the unique rearranged nucleic acid sequences and a distribution of the unique rearranged nucleic acid sequences in the first sample; and

(vii) determining the immune response of the subject to the allograft transplant based on the immune response score.

In some embodiments, the immune response score is calculated from a pre-transplant sample and compared with the immune response score calculated for a post-transplant sample. The immune response score calculated from the post-transplant sample can indicate that the sample has one or more clones having diversity and distribution scores that have changed in a statistically significantly manner in comparison to the diversity and distribution scores of the same clones in a previous sample. In some embodiments, the statistically significant difference can indicate a negative response (e.g., rejection) to the allograft transplant by the subject.

In other embodiments, the immune response score (based on diversity and distribution scores) from a post-transplant sample is compared to a pre-determined threshold (e.g., an average of diversity scores and/or an average of distribution scores) determined from a previous sample from the subject. In another embodiment, the immune response score determined for a post-transplant sample is compared to an immune response score of a control sample. The immune response score calculated from the post-transplant sample can indicate that the sample has one or more clones with diversity and distribution scores that have changed in a statistically significantly manner in comparison to the diversity and distribution scores of the same clones in a control or pre-transplant sample.

### Example 2

### Identification of Transplant-Reactive Clones Indicative of Allograft Transplant Rejection

Refinement of the analysis for allograft rejection can occur by prior identification of the clones from the recipient subject to mediate the rejection process. Two samples from an allograft patient can be taken, such that it can be determined whether the patient has rejected or tolerated the allograft. Clones that were previously low in frequency in a pre-transplant sample and that have expanded in number after the transplant (to a statistically significant degree as compared to the remaining clones in the sample) are identified as transplant-reactive clones (or alloreactive clones). These identified clones are then be specifically tracked and quantified in subsequent diagnostic samples from the same subject or in other subjects.

Prior to transplant, histocompatibility between donor and recipient is interrogated by the use of a mixed lymphocyte reaction (MLR). In one example a “one-way” MLR is used. In a one-way MLR the donor cells are made replication incompetent by irradiation or mitomycin C treatment and are placed in culture with recipient lymphocytes. The culture is then maintained for 5-7 days and, an agent that can be used to quantitate cell division (e.g., BuDR) is added toward the end of the incubation. Robust BuDR incorporation is consistent with a proliferative response of the recipient cells to “foreign” antigens (e.g. a different HL-A antigen or antigens) on the surface of the donor cells. Unstimulated and non-specific mitogen stimulated cells serve as controls.

The one-way MLR assay specifically identifies alloreactive clones (i.e., clones that have expanded from low frequency pre-existing clones) from the peripheral lymphocyte population of a potential allograft recipient. This selection is reproducible and consistent across independent assays performed between the same two donor/recipient pairs.

### Example 3

### Defining the Alloreactive T Cell Repertoire using High-Throughput Sequencing of Mixed Lymphocyte Reaction Culture

Subjects

Human peripheral blood samples were obtained from laboratory volunteers under a protocol following written informed consent approved and supervised by an Institutional Review Board. These healthy volunteers were HLA-typed using molecular methods (reverse sequence specific oligonucleotide probe hybridization).

Mixed Lymphocyte Reaction (MLR) Culture and Alloreactive Responding Cell Isolation

Peripheral blood mononuclear cells (PBMC) were isolated using Ficoll-Hypaque. The recipient cells were labeled with CFSE and the donor cells labeled with PKH26 as described previously [25,26]. The recipients and donors were matched for 1 HLA-DR antigen to mimic the minimum requirement for some clinical transplants [27]. The PKH26 labeled donor cells were also irradiated at 3000 rads. The recipient and donor cells were cultured in bulk in 15% normal AB serum containing RPMI 1640 culture medium (NAB-CM) at 1×10⁶/ml each. After 7 days, these were harvested and the proliferating recipients were then sorted on FACSAria (BD, San Jose, Calif.) by gating on the CFSE dim or negative cells after gating out both CFSE high non-proliferating and the very few PKH26⁺ donor cells that still survived.

In parallel, flow cytometric analysis of the above MLR cultures was performed to determine which subsets of recipient cells proliferated in response to allostimulation, using fluorochrome conjugated monoclonal antibodies. The data were acquired on an FC500 flow cytometer (Beckman-Coulter) and analyzed for cell subsets by gating on the CFSE dim or negative cells after gating out both CFSE high non-proliferating and the very few PKH26⁺ donor cells [25,26]. Additionally, standard 7-day ³H-thymidine incorporation assays were also performed to monitor the strength of the MLR responses as described previously [25,26].

High-Throughput TCRβ Sequencing

Genomic DNA was extracted from cell samples using Qiagen DNeasy Blood extraction Kit (Qiagen, Gaithersburg, Md., USA). The CDR3 region of rearranged TCRβ genes were sequenced; the TCRβ CDR3 region was defined according to the IMGT collaboration [28]. TCRβ CDR3 regions were amplified and sequenced as described above [29,30]. Briefly, a multiplexed PCR method was employed using a mixture of 60 forward primers specific to TCR Vβ gene segments and 13 reverse primers specific to TCR Jβ gene segments. Reads of 87 bp were obtained using the Illumina HiSeq System. Raw HiSeq sequence data were preprocessed to remove errors in the primary sequence of each read, and to compress the data. A nearest neighbor algorithm was used to collapse the data into unique sequences by merging closely related sequences, to remove both PCR and sequencing errors.

PCR Template Abundance Estimation

To estimate the average read coverage per input template in the PCR and sequencing approach, a set of approximately 850 unique types of synthetic TCR analog was employed, comprising each combination of Vβ and Jβ gene segments [29]. These molecules were included in each PCR reaction at very low concentration so that only some types of synthetic template were observed. Using the known concentration of the synthetic template pool, the relationship between the number of observed unique synthetic molecules and the total number of synthetic molecules added to reaction was simulated (at the low concentrations used, this is very nearly one-to-one). These molecules then allowed calculation of the mean number of sequencing reads obtained per molecule of PCR template, and the estimation of the number of T cells in the input material bearing each unique TCR rearrangement for each PCR reaction.

Isolation of the Alloreactive T Cell Repertoire

In order to study the breadth, clonal structure and dynamics of the alloreactive T cell repertoire, a one-way mixed lymphocyte reaction culture was performed using CFSE-labeled recipient cells and PKH26-labeled donor cells on each of three pairs of healthy adult subjects [25,26], with cell culture performed in duplicate. Three months after the first experiment, this cell culture protocol for the same three pairs of subjects was repeated. In total, 18 samples of T cells were generated, comprising six samples from each pair of subjects: uncultured total PBMC and purified proliferating T cells from duplicate MLR, at baseline and after three months.

Figure (FIG.) 1 shows an experimental design for a mixed lymphocyte reaction (MLR) assay followed by high-throughput adaptive immune receptor sequencing. In one example, three pairs of healthy adult subjects were assayed using mixed lymphocyte reaction cultures. For each pair, lymphocytes from a responder subject (Responder Subject #1) were mixed with inactivated lymphocytes from a stimulator subject (Stimulator Subject #1) and cultured in duplicate (Cell cultures 1A and 1B). In this figure, the two subjects are labeled as responder and stimulator, but can also be referred to as “recipient” or “donor,” respectively. Uncultured freshly isolated PBMC from the responder as well as proliferating T cell populations from the duplicate cultures were subjected to high-throughput sequencing. Nine samples in total were sequenced across the three pairs of subjects. Three months later, the experiments were repeated to generate nine more samples for high-throughput TCRβ sequencing.

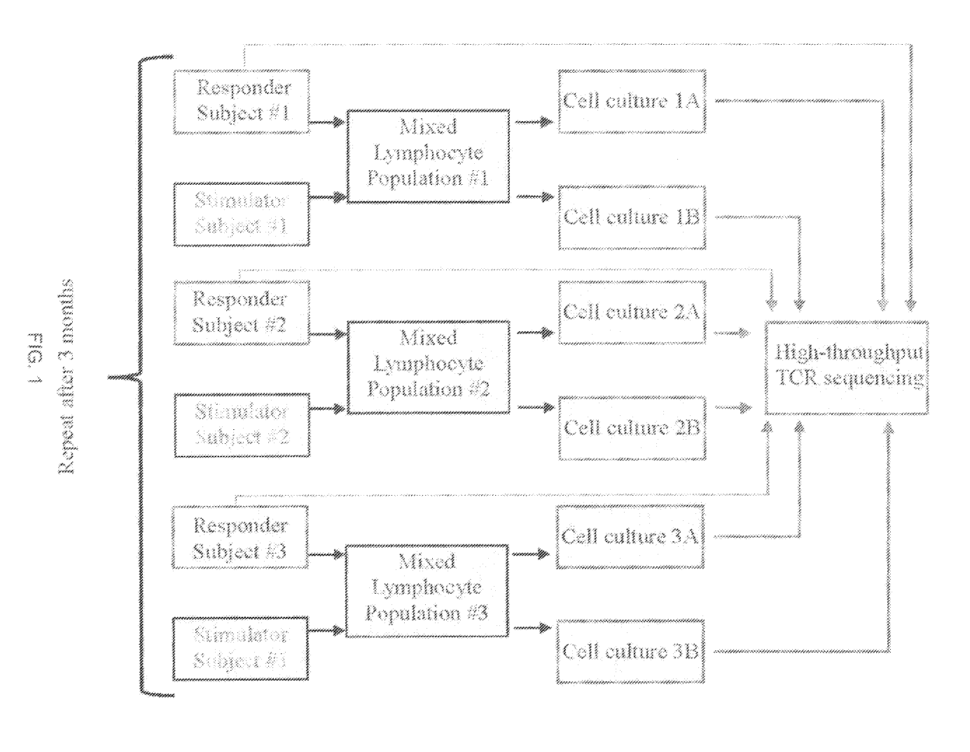

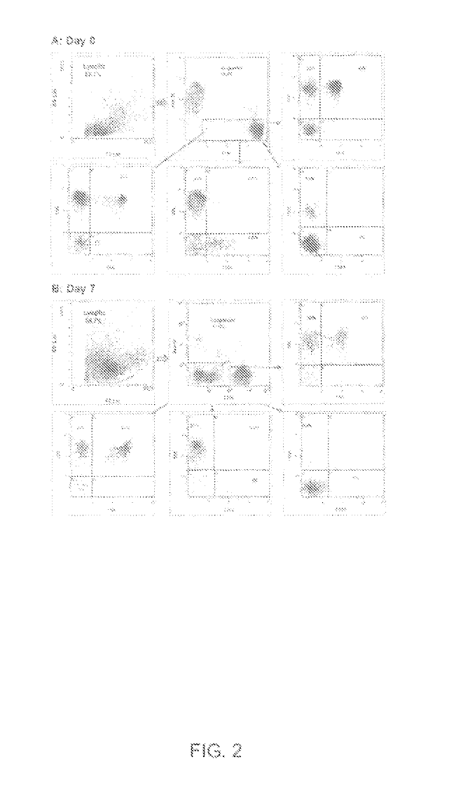

For each MLR reaction, after 7 days the proliferating recipients were sorted by gating on the CFSE dim or negative cells after gating out both CFSE high non-proliferating and the very few PKH26⁺ donor cells that still survived (FIG. 2A). The proliferating cells consisted of 40.3±4.7% CD3⁺CD4⁺ and 57.2±5.1% CD3⁺CD8⁺ T cells as well as minor subset of CD56 NK cells (FIG. 2B). Each population of uncultured PBMC or proliferating T cells was subjected to amplification and high-throughput sequencing of the CDR3 region of TCRβ, which somatically rearranges during T cell maturation and acts as a unique molecular tag for each clonal population of T cells. Sequencing results are presented in Table I (below).

TABLE I
Summary of TCRβ sequencing results

| | T cells | Unique | |
|---|---|---|---|
| | assayed | TCRβ | Sequencing |
| Sample | (estimated) | sequences | reads ^{a} |
| Fresh PBMC sample #1, 0 months | 4,336,812 | 750,211 | 51,160,577 |
| Fresh PBMC sample #2, 0 months | 4,774,312 | 1,375,340 | 46,370,325 |
| Fresh PBMC sample #3, 0 months | 4,016,260 | 991,848 | 33,633,101 |
| Fresh PBMC sample #1, 3 months | 713,990 | 264,159 | 17,437,692 |
| Fresh PBMC sample #2, 3 months | 1,847,987 | 1,046,492 | 23,507,950 |
| Fresh PBMC sample #3, 3 months | 2,197,064 | 1,061,154 | 18,766,880 |
| Proliferated MLR responder #1A, | 1,885,973 | 33,677 | 23,366,016 |
| 0 months | | | |
| Proliferated MLR responder #1B, | 1,997,723 | 33,387 | 26,098,554 |
| 0 months | | | |
| Proliferated MLR responder #2A, | 1,575,201 | 79,174 | 24,704,053 |
| 0 months | | | |
| Proliferated MLR responder #2B, | 1,527,643 | 68,505 | 13,832,785 |
| 0 months | | | |
| Proliferated MLR responder #3A, | 3,372,150 | 58,382 | 37,022,643 |
| 0 months | | | |
| Proliferated MLR responder #3B, | 3,190,902 | 53,316 | 23,126,368 |
| 0 months | | | |
| Proliferated MLR responder #1A, | 640,366 | 57,778 | 12,741,642 |
| 3 months | | | |
| Proliferated MLR responder #1B, | 587,681 | 53,260 | 9,806,707 |
| 3 months | | | |
| Proliferated MLR responder #2A, | 1,022,417 | 68,565 | 10,736,335 |
| 3 months | | | |
| Proliferated MLR responder #2B, | 522,273 | 53,337 | 10,679,864 |
| 3 months | | | |
| Proliferated MLR responder #3A, | 685,126 | 64,615 | 9,788,942 |
| 3 months | | | |
| Proliferated MLR responder #3B, | 760,990 | 67,586 | 10,999,866 |
| 3 months | | | |
| | 35,654,870 | 6,180,786 | 403,780,300 |

^{a} the total number of 87-bp sequencing reads generated.

Size of the Alloreactive T Cell Repertoire

To determine the number of T cell clonal lineages involved in the alloreactive T cell response, the number of unique CDR3 sequences observed in the proliferated T cell samples was analyzed in comparison to uncultured bulk T cells from the same subjects. The alloreactive T cell clones were defined as those observed in at least 10 cells in the proliferated sample and unobserved in the uncultured T cell sample, or T cells whose frequency in the proliferated sample was at least ten-fold higher than in the uncultured T cell sample. Two sets of alloreactive T cell clones were defined: low-abundance alloreactive clones (below the threshold of detection in the subject's baseline T cell repertoire) and high-abundance alloreactive clones (present at measurable frequency in the subject's baseline T cell repertoire). On average, 14,000 alloreactive T cell clones were observed in each experiment; 84% of alloreactive T cell clones were low-abundance before proliferation, but in total low-abundance clones made up 40% and high-abundance clones made up 60% of the alloreactive T cell repertoire when weighting by post-proliferation clonal abundance (See Table II below). While the number of proliferated low-abundance clones varied considerably, variation in the number of high-abundance (thus, presumably antigen-experienced) T cell clones between subjects was much smaller, at about 2,000 clones in each of the six experiments. These data indicated that thousands of different clonal populations of T cells comprise the alloreactive T cell repertoire.

TABLE II
Size of the alloreactive T cell repertoire

| | Mean | | % of proliferated |
|---|---|---|---|
| | (N = 6) | SD | T cells |
| Number of alloreactive clones | 13750 | 6823 | 100% |
| Low-abundance pre-culture ^{a} | 11610 | 6494 | 40.0% |
| High-abundance pre-culture ^{b} | 2140 | 539 | 60.0% |

^{a} unobserved in pre-culture sample and ≧10 T cells after MLR
^{b} present in pre-culture sample and ≧10× enriched after MLR.

Reproducibility of the Alloreactive T Cell Repertoire

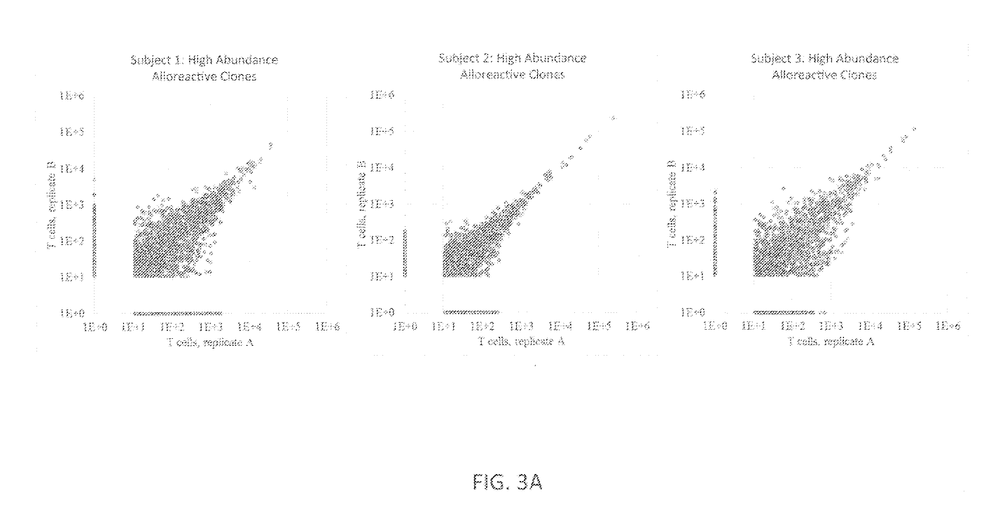

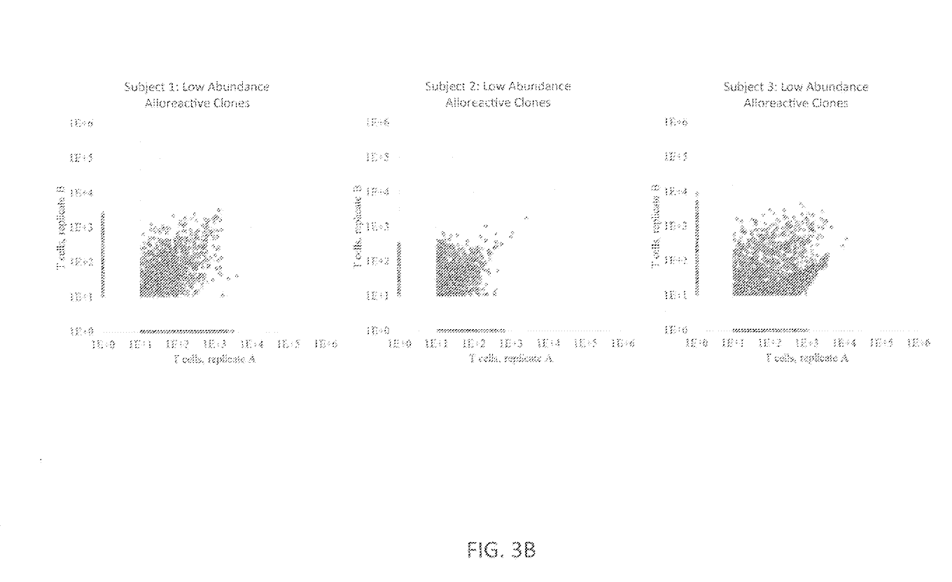

To assay the consistency of the alloreactive T cell repertoire, the persistence of each T cell clone was examined. After defining high-abundance and low-abundance alloreactive T cells, the set of alloreactive T cell clones generated in duplicate cell culture experiments was compared, shown in FIGS. 3A and 3B respectively. In each subject, essentially all clones that were highly expanded in proliferated cell culture assorted to the high-abundance subset (i.e., were present at appreciable frequency in the peripheral T cell repertoire to begin with). Reproducibility between duplicate cell culture experiments was high among this set of abundant and highly alloreactive T cell clones (average r² among three subjects=0.96), indicating that when presented with identical stimuli, these clonal populations of T cells responded in a very reproducible manner.

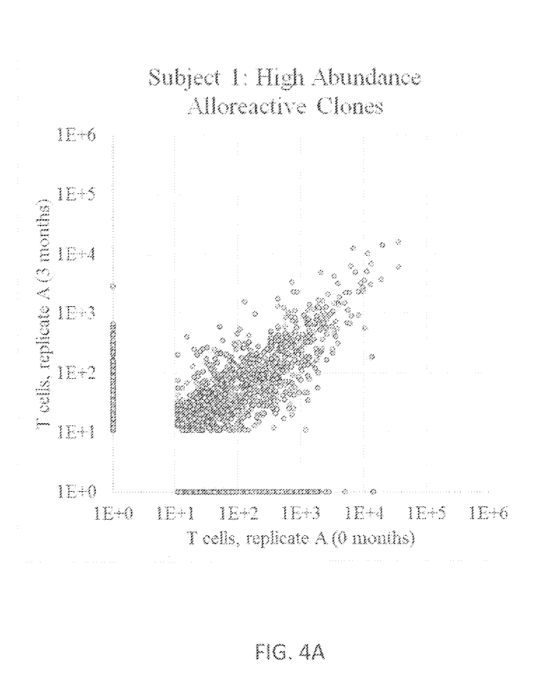

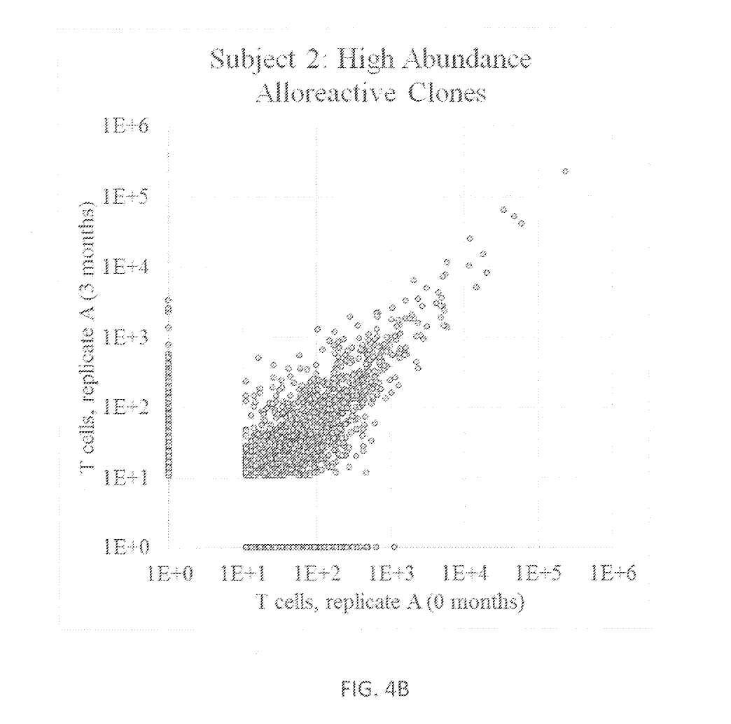

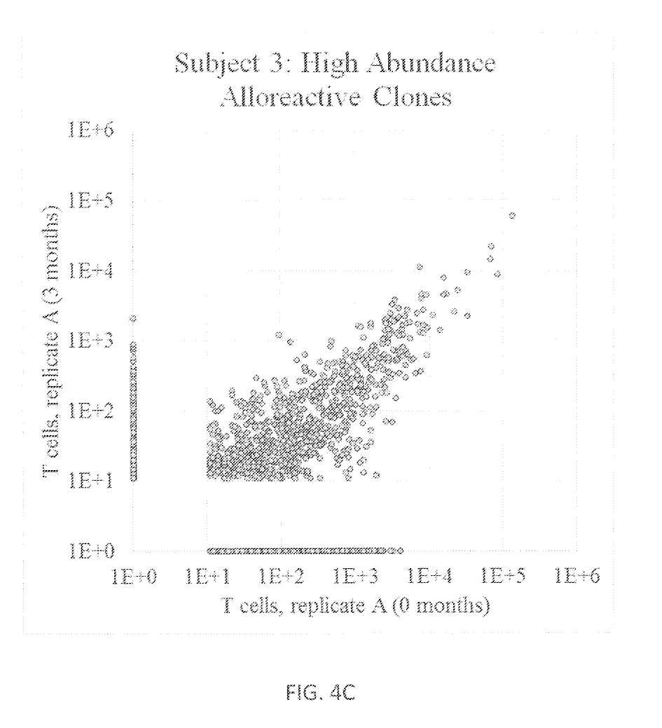

Since the replicate cell culture experiments did not address the stability of the alloreactive T cell repertoire over time, the T cell isolation and duplicate MLR experiments were repeated with the same three pairs of subjects three months after our initial experiment. Specifically, it was hypothesized that high-abundance alloreactive clones, which were presumed to represent memory T cells due to their frequency in the peripheral T cell repertoire, should be stable over time and thus should remain in the alloreactive T cell compartment. FIGS. 4A-4C show the high-abundance T cell repertoire after three months in each pair of subjects (subjects 1, 2, and 3). Many T cell clones identified as part of the high-abundance alloreactive T cell repertoire at baseline were observed in the high-abundance alloreactive T cell repertoire three months later, at similar clonal frequencies (FIGS. 4A-4C; average r²=0.78).

To quantify similarity between sets of T cells, a TCR overlap metric was calculated (the proportion of T cells belonging to clones found in both samples) [29]. Table III below presents the TCR overlap between duplicate cell culture experiments and between experiments spaced three months apart. While duplicate cell culture experiments generated more concordant sets of alloreactive T cell clones than experiments from different time-points, overlap between different time-points was nonetheless quite high (mean overlap=0.97 for duplicate experiments vs. 0.87 across time-points). It was hypothesized that the lower overlap over time might be due to the emergence of naïve T cell clones of exceptional size which would not be expected to persist in the periphery and/or the noise in the estimation of absolute cellular abundance could have caused a subset of low-abundance clones to be erroneously classified as high-abundance in the experiment [31-33].

The low-abundance alloreactive T cell clones, however, showed lower reproducibility between duplicate cell culture experiments (Table III, bottom) and appeared to be considerably more transient. Comparisons between biological duplicates were much more concordant than comparisons between time-points (mean overlap=0.55 for duplicate experiments vs. 0.10 across time-points). Several hypotheses may explain why T cell clones were not reproducibly found in the low-abundant alloreactive T cell compartment; first, the lower overlap between biological replicates is mostly due to sample error (most unique T cell lineages are at very low abundance, and a T cell clone could not reliably be found in two biological replicates unless it is present in at least several cells); second, the even lower reproducibility after three months can be attributed to a preponderance of newly emerged naïve T cell clones among this subset; lastly, these clones may represent memory T cell populations that did not persist at detectable levels in the periphery over the intervening time [31-33].

TABLE III
TCR overlap between biological & temporal replicate mixed lymphocyte
culture experiments

| | Biological replicates | Temporal replicates |
|---|---|---|
| | (N = 2) ^{a} | (N = 4) ^{b} |
| High-abundance pre-culture ^{c} | | |
| Subject 1 | 0.96 | 0.78 |
| Subject 2 | 0.98 | 0.93 |
| Subject 3 | 0.98 | 0.89 |
| Average | 0.97 | 0.87 |
| Low-abundance pre-culture ^{d} | | |
| Subject 1 | 0.54 | 0.15 |
| Subject 2 | 0.43 | 0.06 |
| Subject 3 | 0.67 | 0.08 |
| Average | 0.55 | 0.10 |

^{a} MLR Cultured in duplicate
^{b} MLR performed at three months apart
^{c} Present in pre-culture sample and ≧10× enriched after MLR
^{d} Unobserved in pre-culture sample and ≧10 T cells after MLR.

Taken together, the TCR repertoire analysis described above was highly sensitive and reproducible. Further, the results indicated that a majority of the alloreactivity observed between three pairs of healthy adults was attributable to a set of several thousand T cell clones, present at reasonably high frequency in the peripheral T cell repertoire, whose alloreactive potential remained stable over at least several months. The screening algorithm (requiring a T cell clone to represent a 10× higher proportion of the proliferated than the fresh sample) should ensure that only a minimal number of nonspecifically-proliferating clones are identified.

The application of the methods of the invention to transplantation could have a positive impact in the clinical management of patients. This would be achieved by performing donor-specific MLR at transplant to pre-define the donor-reactive T cell repertoire, and then tracking their presence, abundance and dynamics in recipient primary tissues (e.g. peripheral blood, allograft biopsies, urine) during the post-transplant period. Such an approach has applications for the technology in both living donor and deceased donor transplants. The alloreactive T cell repertoire could thus be combined with post-transplant immune profiling in the recipient peripheral blood for non-invasive monitoring of cellular.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

1. Adams A B, Williams M A, Jones T R, Shirasugi N, Durham M M, et al. (2003) Heterologous immunity provides a potent barrier to transplantation tolerance. J Clin Invest 111: 1887-1895.

2. Brook M O, Wood K J, Jones N D (2006) The impact of memory T cells on rejection and the induction of tolerance. Transplantation 82: 1-9.

3. D'Orsogna L J, Roelen D L, Doxiadis, II, Claas F H (2012) TCR cross-reactivity and allorecognition: new insights into the immunogenetics of allorecognition. Immunogenetics 64: 77-85.

4. Burrows S R, Khanna R, Silins S L, Moss D J (1999) The influence of antiviral T-cell responses on the alloreactive repertoire. Immunol Today 20: 203-207.

5. Welsh R M, Selin L K (2002) No one is naive: the significance of heterologous T-cell immunity. Nat Rev Immunol 2: 417-426.

6. Borbulevych O Y, Piepenbrink K H, Gloor B E, Scott D R, Sommese R F, et al. (2009) T cell receptor cross-reactivity directed by antigen-dependent tuning of peptide-MHC molecular flexibility. Immunity 31: 885-896.

7. Colf L A, Bankovich A J, Hanick N A, Bowerman N A, Jones L L, et al. (2007) How a single T cell receptor recognizes both self and foreign MHC. Cell 129: 135-146.

8. Ely L K, Burrows S R, Purcell A W, Rossjohn J, McCluskey J (2008) T-cells behaving badly: structural insights into alloreactivity and autoimmunity. Curr Opin Immunol 20: 575-580.

9. Griesemer A D, Sorenson E C, Hardy M A (2010) The role of the thymus in tolerance. Transplantation 90: 465-474.

10. Macedo C, Orkis E A, Popescu I, Elinoff B D, Zeevi A, et al. (2009) Contribution of naive and memory T-cell populations to the human alloimmune response. Am J Transplant 9: 2057-2066.

11. Marrack P, Kappler J (1988) T cells can distinguish between allogeneic major histocompatibility complex products on different cell types. Nature 332: 840-843.

12. Schild H, Rotzschke O, Kalbacher H, Rammensee H G (1990) Limit of T cell tolerance to self proteins by peptide presentation. Science 247: 1587-1589.

13. Argaet V P, Schmidt C W, Burrows S R, Silins S L, Kurilla M G, et al. (1994) Dominant selection of an invariant T cell antigen receptor in response to persistent infection by Epstein-Barr virus. J Exp Med 180: 2335-2340.

14. Burrows S R, Khanna R, Burrows J M, Moss D J (1994) An alloresponse in humans is dominated by cytotoxic T lymphocytes (CTL) cross-reactive with a single Epstein-Barr virus CTL epitope: implications for graft-versus-host disease. J Exp Med 179: 1155-1161.

15. Burrows S R, Silins S L, Moss D J, Khanna R, Misko I S, et al. (1995) T cell receptor repertoire for a viral epitope in humans is diversified by tolerance to a background major histocompatibility complex antigen. J Exp Med 182: 1703-1715.

16. Gras S, Burrows S R, Kjer-Nielsen L, Clements C S, Liu Y C, et al. (2009) The shaping of T cell receptor recognition by self-tolerance. Immunity 30: 193-203.

17. Mifsud N A, Nguyen T H, Tait B D, Kotsimbos T C (2010) Quantitative and functional diversity of cross-reactive EBV-specific CD8+ T cells in a longitudinal study cohort of lung transplant recipients. Transplantation 90: 1439-1449.

18. Amir A L, D'Orsogna L J, Roelen D L, van Loenen M M, Hagedoorn R S, et al. (2010) Allo-HLA reactivity of virus-specific memory T cells is common. Blood 115: 3146-3157.

19. Danziger-Isakov L, Cherkassky L, Siegel H, McManamon M, Kramer K, et al. (2010) Effects of influenza immunization on humoral and cellular alloreactivity in humans. Transplantation 89: 838-844.

20. D'Orsogna L J, Roelen D L, Doxiadis, II, Claas F H (2011) Screening of viral specific T-cell lines for HLA alloreactivity prior to adoptive immunotherapy may prevent GvHD. Transpl Immunol 24: 141.

21. D'Orsogna L J, van Besouw N M, van der Meer-Prins E M, van der Pol P, Franke-van Dijk M, et al. (2011) Vaccine-induced allo-HLA-reactive memory T cells in a kidney transplantation candidate. Transplantation 91: 645-651.

22. Wang T, Chen L, Ahmed E, Ma L, Yin D, et al. (2008) Prevention of allograft tolerance by bacterial infection with Listeria monocytogenes. J Immunol 180: 5991-5999.

23. Bain B, Lowenstein L (1964) GENETIC STUDIES ON THE MIXED LEUKOCYTE REACTION. Science 145: 1315-1316.

24. Bain B, Vas M R, Lowenstein L (1964) THE DEVELOPMENT OF LARGE IMMATURE MONONUCLEAR CELLS IN MIXED LEUKOCYTE CULTURES. Blood 23: 108-116.

25. Levitsky J, Leventhal J R, Miller J, Huang X, Chen L, et al. (2012) Favorable effects of alemtuzumab on allospecific regulatory T-cell generation. Human Immunology 73: 141-149.

26. Levitsky J, Miller J, Huang X, Chandrasekaran D, Chen L, et al. (2013) Inhibitory Effects of Belatacept on Allospecific Regulatory T-Cell Generation in Humans. Transplantation 96: 689-696 610.1097/TP.1090b1013e31829f31607.

27. Mathew J M, Garcia-Morales R O, Carreno M, Jin Y, Fuller L, et al. (2003) Immune responses and their regulation by donor bone marrow cells in clinical organ transplantation. Transplant Immunology 11: 307-321.

28. Yousfi Monod M, Giudicelli V, Chaume D, Lefranc M P (2004) IMGT/JunctionAnalysis: the first tool for the analysis of the immunoglobulin and T cell receptor complex V-J and V-D-J JUNCTIONs. Bioinformatics 20 Suppl 1: i379-385.

29. Emerson R O, Sherwood A M, Rieder M J, Guenthoer J, Williamson D W, et al. (2013) High-throughput sequencing of T-cell receptors reveals a homogeneous repertoire of tumour-infiltrating lymphocytes in ovarian cancer. J Pathol 231: 433-440.

30. Robins H S, Campregher P V, Srivastava S K, Wacher A, Turtle C J, et al. (2009) Comprehensive assessment of T-cell receptor beta-chain diversity in alphabeta T cells. Blood 114: 4099-4107.

31. Jenkins M K, Chu H H, McLachlan J B, Moon J J (2010) On the composition of the preimmune repertoire of T cells specific for Peptide-major histocompatibility complex ligands. Annu Rev Immunol 28: 275-294.

32. Jenkins M K, Moon J J (2012) The role of naive T cell precursor frequency and recruitment in dictating immune response magnitude. J Immunol 188: 4135-4140.

33. Surh C D, Sprent J (2008) Homeostasis of naive and memory T cells. Immunity 29: 848-862.

What is claimed is:

1. A method for defining an alloreactive adaptive immune cell repertoire, comprising:
obtaining a first sample comprising lymphocytes of a recipient subject at a time point prior to an allograft, and a second sample comprising lymphocytes of a donor subject;
obtaining a mixed lymphocyte reaction (MLR) sample comprising a mixture of proliferating lymphocytes from said first and second samples;
generating an adaptive immune profile of adaptive immune cell clones comprising unique rearranged CDR3-encoding region DNA sequences for the first sample and the MLR sample; and
identifying one or more alloreactive clones in the adaptive immune profile that are expanded in frequency of occurrence in said MLR sample compared to said first sample.

2. The method of claim 1, further comprising determining a presence or an absence of the one or more identified alloreactive clones in a post-allograft sample obtained from said recipient subject after the transplant.

3. The method of claim 2, further comprising determining a frequency of occurrence of the one or more identified alloreactive clones in a post-allograft sample, wherein the frequency of occurrence of the identified alloreactive clone is predictive of an immune response of the recipient subject to the allograft.

4. The method of claim 1, wherein generating an adaptive immune profile of adaptive immune cell clones comprises:
obtaining rearranged DNA templates comprising T cell receptor (TCR) or Immunoglobulin (Ig) CDR3-encoding regions from the lymphocytes in the first sample and MLR sample;
amplifying the rearranged DNA templates in a single multiplex PCR to produce a plurality of rearranged DNA amplicons;
sequencing said plurality of rearranged DNA amplicons to produce a plurality of rearranged DNA sequences; and
determining a number of unique rearranged CDR3-encoding DNA sequences in the sample.

5. The method of claim 1, further comprising determining a frequency of occurrence of each unique rearranged CDR3-encoding DNA sequence in the first sample and MLR sample.

6. The method of claim 1, wherein the first sample comprising lymphocytes comprise T cells.

7. The method of claim 1, wherein the first sample comprising lymphocytes comprise B cells.

8. The method of claim 1, wherein the second sample comprising lymphocytes comprise T cells.

9. The method of claim 1, wherein the second sample comprising lymphocytes comprise B cells.

10. The method of claim 1, wherein the MLR sample comprises T cells.

11. The method of claim 1, wherein the MLR sample comprises B cells.

12. The method of claim 1, wherein identifying one or more alloreactive clones comprises identifying a clone that has a frequency of occurrence below a first predetermined threshold in the first sample and has a frequency of occurrence that is greater than a second predetermined threshold in the MLR sample.

13. The method of claim 1, wherein the clone is not observed in the first sample.

14. The method of claim 1, wherein the second predetermined threshold is n-fold greater than the first predetermined threshold.

15. The method of claim 1, wherein identifying one or more alloreactive clones comprises identifying a clone that has an n-fold higher frequency of occurrence in the MLR sample than the frequency of occurrence of the clone in the first sample.

16. The method of claim 14 or 15, wherein n is 2 or greater, or 3 or greater, or 4 or greater, or 5 or greater, or 6 or greater, or 7 or greater or 8 or greater, or 9 or greater, or 10 or greater.

17. The method of claim 1, wherein identifying one or more alloreactive clones comprises identifying a clone that has a statistically significantly higher frequency of occurrence in the MLR sample than in the first sample.

18. The method of claim 1, further comprising characterizing an alloreactive clone as a low-abundance alloreactive clone if the clone has a frequency of occurrence below a predetermined threshold of detection in the sample.

19. The method of claim 1, further comprising characterizing an alloreactive clone as a high-abundance alloreactive clone if the clone has a frequency of occurrence that is greater than a predetermined threshold for a baseline frequency in the sample.

20. The method of claim 1, further comprising characterizing an alloreactive clone as a high-abundance alloreactive clone if the clone has a frequency of occurrence that is statistically significantly greater than a mean frequency of clones in the sample.

21. The method of claim 1, wherein the first sample or the second sample comprises a blood sample.

22. The method of claim 1, wherein the first sample or the second sample comprises a lymphocyte sample.

23. The method of claim 2, wherein the post-allograft sample comprises a blood sample.

24. The method of claim 2, wherein the post-allograft sample comprises a urine sample.

25. The method of claim 2, wherein the post-allograft sample comprises a tissue sample.

26. The method of claim 2, further comprising determining that the allograft is rejected based on the frequency of occurrence of at least one identified alloreactive clone in the post-allograft sample.

27. The method of claim 2, further comprising determining that the allograft is tolerated based on the frequency of occurrence of at least one identified alloreactive clone in the post-allograft sample.

28. The method of claim 1, further comprising determining a measure of overlap of alloreactive adaptive immune cell clones between first sample and the MLR sample.

29. The method of claim 1, further comprising determining a treatment for the recipient subject based on the identified one or more alloreactive clones in the adaptive immune profile.

30. The method of claim 1, further comprising screening the recipient subject for an allograft based on the identified one or more alloreactive clones in the adaptive immune profile.

31. The method of claim 1, further comprising determining whether an alloreactive adaptive immune cell clone is persistent between two samples.

32. The method of claim 1, further comprising determining whether an alloreactive adaptive immune cell clone is transient between two samples.

33. A method for determining an immune response of a subject undergoing an allograft transplant, comprising:
determining the sequences of a plurality of unique rearranged nucleic acid sequences, each of said plurality of unique rearranged nucleic acid sequences encoding an adaptive immune receptor (AIR) polypeptide, in a first sample obtained from said subject at a first time point prior to said allograft transplant;
determining a first immune response score for said first sample based on a diversity of said unique rearranged nucleic acid sequences and a distribution of said unique rearranged nucleic acid sequences in said first sample; and
determining an immune response of said subject to said allograft transplant based on said first immune response score.

34. The method of claim 33, wherein determining the first immune response score comprises quantifying an AIR sequence diversity score for said first sample based on a total number of unique rearranged DNA sequences determined from nucleic acid sequence information from said first sample.

35. The method of claim 34, wherein quantifying said AIR sequence diversity score comprises determining a total number of unique clones in said first sample.

36. The method of claim 33, wherein determining a first immune response score comprises quantifying an AIR sequence distribution score for said first sample by calculating a frequency of occurrence of each unique rearranged DNA sequence as a percentage of a total number of observed rearranged sequences determined from nucleic acid sequence information from said first sample.

37. The method of claim 33, wherein determining a first immune response score comprises:
quantifying an AIR sequence diversity score for said first sample based on a total number of unique rearranged DNA sequences determined from nucleic acid sequence information from said first sample; and
quantifying an AIR sequence distribution score for said first sample by calculating a frequency of occurrence of each unique rearranged DNA sequence as a percentage of a total number of observed rearranged sequences determined from nucleic acid sequence information from said first sample

38. The method of claim 33, further comprising:
comparing said first immune response score for said first sample to a second immune response score determined for a second sample obtained from said subject at a second time point after said allograft transplant.

39. The method of claim 38, wherein a statistically significant difference between the first immune response score and the second immune response score is predictive of rejection of said allograft transplant by said subject.

40. The method of claim 38, further comprising determining that said subject has tolerated the allograft transplant based on said comparison of said first immune response score and said second immune response score wherein no difference or a statistically insignificant difference indicates said subject has tolerated the allograft.

41. The method of claim 33, further comprising determining a frequency of occurrence of one or more clones in said first sample at said first time point and a frequency of occurrence of one or more clones in said second sample at said second time point after said allograft transplant.

42. The method of claim 41, further comprising identifying one or more clones from said second sample that have a frequency of occurrence that is statistically significantly greater than an average frequency of occurrence of said unique rearranged nucleic acid sequences in said second sample.

43. The method of claim 41, further comprising identifying one or more clones in said second sample that have a frequency of occurrence that is statistically significantly greater than a top quartile of frequency of occurrence of said unique rearranged nucleic acid sequences in said second sample.

44. The method of claim 41, further comprising identifying one or more clones in said second sample that have a frequency of occurrence that is statistically significantly higher than 50% of frequencies of occurrence of said unique rearranged nucleic acid sequences in said second sample.

45. The method of any one of claims 41-44, further comprising determining that said one or more clones is an expanded clone, wherein said expanded clone has increased in frequency of occurrence from a low frequency clone in said first sample to a high frequency clone in said second sample.

46. The method of claim 45, wherein a presence of said one or more expanded clones in said second sample is indicative of a rejection of said allograft transplant by said subject.

47. The method of claim 46, further comprising measuring a frequency of occurrence of said one or more expanded clones in subsequent samples obtained from said subject after said allograft transplant.

48. The method of any one of claims 1-47, wherein said first sample and/or said second sample comprise a tissue sample.

49. The method of claim 48, wherein said tissue sample comprises a tissue sample from said allograft transplant.

50. The method of any one of claims 1-47, wherein said first sample and/or said second sample comprise a circulating blood mononuclear cell fraction.

51. The method of any one of claims 1-47, wherein said first sample and/or said second sample comprise cells collected from urinary sediment.

52. The method of any one of claims 1-51, wherein said nucleic acid sequences comprise genomic DNA sequences.

53. The method of any one of claims 1-51, wherein said nucleic acid sequences comprise RNA sequences.

54. The method of any one of claims 1-51, wherein said nucleic acid sequences comprise complementary DNA (cDNA) sequences.

55. The method of any one of claims 1-54, further comprising amplifying nucleic acid sequences obtained from a first sample or a second sample comprising lymphoid cells of said subject in a multiplexed polymerase chain reaction (PCR) assay to produce a plurality of amplified nucleic acid sequences using (1) a plurality of AIR V-segment oligonucleotide primers and (2) either a plurality of AIR J-segment oligonucleotide primers or a plurality of AIR C-segment oligonucleotide primers.

56. The method of claim 55, wherein said plurality of AIR V-segment oligonucleotide primers are each independently capable of specifically hybridizing to at least one polynucleotide encoding a mammalian AIR V-region polypeptide, wherein each AIR V-segment oligonucleotide primer comprises a nucleotide sequence of at least 15 contiguous nucleotides that is complementary to at least one functional AIR-encoding gene segment, wherein said plurality of AIR V-segment oligonucleotide primers specifically hybridize to substantially all functional AIR V-encoding gene segments that are present in said first or second samples;
wherein said plurality of J-segment oligonucleotide primers are each independently capable of specifically hybridizing to at least one polynucleotide encoding a mammalian AIR J-region polypeptide, wherein each J-segment primer comprises a nucleotide sequence of at least 15 contiguous nucleotides that is complementary to at least one functional AIR J-encoding gene segment, wherein said plurality of J-segment primers specifically hybridize to substantially all functional AIR J-encoding gene segments that are present in said first or second samples;
wherein said plurality of C-segment oligonucleotide primers are each independently capable of specifically hybridizing to at least one polynucleotide encoding a mammalian AIR C-region polypeptide, wherein each C-segment primer comprises a nucleotide sequence of at least 15 contiguous nucleotides that is complementary to at least one functional AIR C-encoding gene segment, wherein the plurality of C-segment primers specifically hybridize to substantially all functional AIR C-encoding or gene segments that are present in said first or second samples; and
wherein (1) said plurality of AIR V-segment oligonucleotide primers, and (2) either said plurality of AIR J-segment oligonucleotide primers and said plurality of AIR C-segment oligonucleotide primers are capable of promoting amplification in said multiplex PCR of substantially all rearranged AIR CDR3-encoding regions in said first or second samples to produce a plurality of amplified rearranged nucleic acid molecules sufficient to quantify the full diversity of said AIR CDR3-encoding region in said first or second samples.

57. The method of claim 56, wherein each functional AIR V-encoding gene segment comprises a V gene recombination signal sequence (RSS) and each functional AIR J-encoding gene segment comprises a J gene RSS, wherein each amplified rearranged DNA molecule comprises (i) at least 10, 20, 30 or 40 contiguous nucleotides of a sense strand of said AIR V-encoding gene segment, wherein said at least 10, 20, 30 or 40 contiguous nucleotides are situated 5′ to said V gene RSS and (ii) at least 10, 20 or 30 contiguous nucleotides of a sense strand of said AIR J-encoding gene segment, wherein said at least 10, 20 or 30 contiguous nucleotides are situated 3′ to said J gene RSS.

58. The method of claim 55, wherein each amplified rearranged nucleic acid molecule is less than 1500 nucleotides in length.

59. The method of claim 58, wherein each amplified rearranged nucleic acid molecule is less than 1000 nucleotides in length.

60. The method of claim 59, wherein each amplified rearranged nucleic acid molecule is less than 600 nucleotides in length.

61. The method of claim 60, wherein each amplified rearranged nucleic acid molecule is less than 500 nucleotides in length.

62. The method of claim 61, wherein each amplified rearranged nucleic acid molecule is 400 nucleotides in length.

63. The method of claim 62, wherein each amplified rearranged nucleic acid molecule is less than 300 nucleotides in length.

64. The method of claim 63, wherein each amplified rearranged nucleic acid molecule is less than 200 nucleotides in length.

65. The method of claim 64, wherein each amplified rearranged nucleic acid molecule is less than 100 nucleotides in length.

66. The method of claim 55, wherein each amplified rearranged nucleic acid molecule is between 50-600 nucleotides in length.

67. The method of claim 33, further comprising determining a histocompatibility between a donor subject and a recipient subject using a mixed lymphocyte reaction (MLR).

68. The method of claim 67, further comprising identifying clones from a biological sample of said recipient subject using an MLR assay, wherein said clones are predicted to expand in frequency of occurrence after said allograft transplant.

69. The method of claim 68, wherein said biological sample comprises a peripheral T-cell population.

70. The method of claim 33, further comprising further comprising providing a treatment for said subject based on said determined immune response.

71. The method of any one of claims 1-70, wherein said adaptive immune receptor (AIR) polypeptide is a mammalian AIR polypeptide and is selected from a T cell receptor-gamma (TCRG) polypeptide, a T cell receptor-beta (TCRB) polypeptide, a T cell receptor-alpha (TCRA) polypeptide, a T cell receptor-delta (TCRD) polypeptide, an immunoglobulin heavy-chain (IGH) polypeptide, and an immunoglobulin light-chain (IGL) polypeptide.

72. The method of claim 71, wherein said IGH polypeptide is selected from an IgM, an IgA polypeptide, an IgG polypeptide, an IgD polypeptide and an IgE polypeptide.

73. The method of claim 71, wherein said IGL polypeptide is selected from an IGL-lambda polypeptide and an IGL-kappa polypeptide.

74. The method of claim 71, wherein said mammalian AIR polypeptide is a human AIR polypeptide.

75. The method of claim 71, wherein said mammalian AIR polypeptide is selected from a non-human primate AIR polypeptide, a rodent AIR polypeptide, a canine AIR polypeptide, a feline AIR polypeptide and an ungulate AIR polypeptide.
